(19) United States
(12) Patent Application Publication
(10) Pub. No.: US 2022/0137260 A1
(43) Pub. Date: May 5, 2022

(54) POLYMERIZABLE COMPOSITION, COMPOUND, POLYMER, RESIN COMPOSITION, ULTRAVIOLET CUT FILM, AND LAMINATE

(71) Applicant: FUJIFILM CORPORATION, Tokyo (JP)

(72) Inventors: Yoshihiro JIMBO, Shizuoka (JP); Naoyuki MOROOKA, Shizuoka (JP)

(21) Appl. No.: 17/575,651

(22) Filed: Jan. 14, 2022

Related U.S. Application Data
(63) Continuation of application No. PCT/JP2020/024476, filed on Jun. 22, 2020.

(30) Foreign Application Priority Data
Aug. 15, 2019 (JP) 2019-149171

(51) Int. Cl. G02B 1/04; C08F 220/28; C08J 5/18; C07D 263/56; G02B 5/20

(52) U.S. Cl. CPC G02B 1/04; C08F 220/281; C08J 2333/14; C07D 263/56; G02B 5/208; C08J 5/18

(57) ABSTRACT

Provided are a polymerizable composition containing a compound represented by General Formula (I-1) or General Formula (I-2) and a polymerizable compound, applications of the polymerizable composition, a compound represented by General Formula (II), and applications of the compound. In General Formula (I-1), General Formula (I-2), and General Formula (II), R¹ represents a hydrogen atom, an alkyl group, or the like, R² and R³ each represent a hydrogen atom, an alkyl group, or the like, R⁴ and R⁵ each represent an electron-withdrawing group, and A represents a 5- or 6-membered ring or the like. In General Formula (I-1), D represents O, S, or N-E, and E represents an alkyl group. At least one of R¹, R², R³, R⁴, R⁵, or A in General Formula (II) contains a substituent selected from the group consisting of General Formula (III) and General Formula (IV).

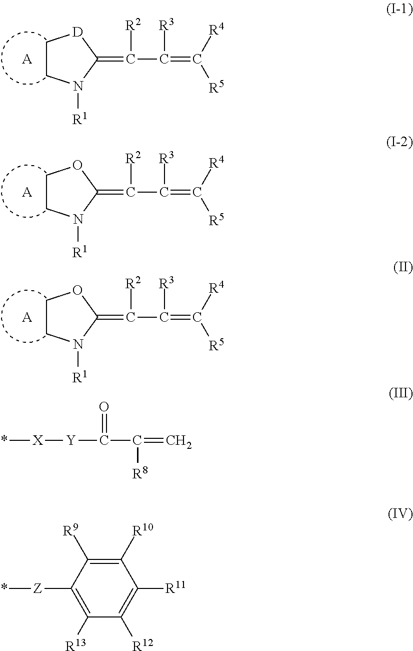

### CROSS-REFERENCE TO RELATED APPLICATIONS

This application is a continuation application of International Application No. PCT/JP2020/024476, filed Jun. 22, 2020, which is incorporated herein by reference. Further, this application claims priority from Japanese Patent Application No. 2019-149171, filed Aug. 15, 2019, which is incorporated herein by reference.

### BACKGROUND OF THE INVENTION

### 1. Field of the Invention

The present disclosure relates to a polymerizable composition, a compound, a polymer, a resin composition, an ultraviolet cut film, and a laminate.

### 2. Description of the Related Art

Attention is being paid to the influences exerted on the retina by the light of various wavelengths that directly comes into the human eye. Especially, ultraviolet rays or blue light damages the retina and sometimes leads to eye diseases, which raises concern.

In using an apparatus comprising a display, such as an image display apparatus including a liquid crystal display device and an electroluminescent display or a small terminal including a smartphone and a tablet terminal, the user sees the screen of the display comprising a light source. In recent years, attention has been paid to the influences exerted on the retina by ultraviolet rays and blue light in a case where an image display device, a small terminal, or the like is used for a long time. Therefore, there is a desire for suppressing the transmission of light having a wavelength of 300 nm to 500 nm as ultraviolet light in a long wavelength region and visible light in a short wavelength region so that the influences on the user's eye are reduced.

As one of the measures to reduce the influences exerted on the user's eye by the light having a wavelength of 300 nm to 500 nm, an attempt has been made so that blue light is absorbed into a protective sheet for display, and that the influences on the user's eye are reduced.

Furthermore, as another measure, an attempt has been made so that ultraviolet rays and blue light is absorbed into spectacle lenses or contact lenses the user wears in watching the screen of the display comprising a light source and that the influences on the user's eye are reduced.

As a protective sheet to absorb blue light, a protective sheet containing a yellow colorant having a maximal absorption wavelength at 400 nm to 500 nm has been proposed (see JP2015-87690A).

In addition, a blue light cut resin composition which contains a yellow colorant and a purple colorant so as not to impair the natural color tone and a resin molded article which is formed of the blue light cut resin composition have been proposed. (see JP2015-17152A).

In the related art, a resin composition containing an ultraviolet absorber has problems such as the occurrence of bleed-out, outflow, precipitation, and the like of the ultraviolet absorber. As a solution to these problems, the ultraviolet absorber is covalently bonded to the resin and immobilized so that the performance deterioration resulting from the bleed-out, outflow, precipitation, and the like of the ultraviolet absorber is suppressed.

A case has been reported in which an ultraviolet absorber into which a polymerizable group is introduced is introduced into a resin composition for the purpose described above (for example, see JP2000-123621A and WO2019/073869A).

### SUMMARY OF THE INVENTION

The yellow colorant contained in the protective sheet described in JP2015-87690A has absorption in a wavelength region useful for cutting off blue light. However, this colorant has a broad absorption curve in a wavelength range of 400 nm to 500 nm, and sometimes absorbs light of a wavelength region longer than the maximal absorption wavelength. Therefore, unfortunately, the protective sheet has a reddish tone, and the obtained color reproducibility of the display image is insufficient.

The blue light cut resin composition described in JP2015-17152A is required to use two kinds of colorants of different color tones in combination. Furthermore, even though being able to cut off blue light, this resin composition also has absorption in a visible light region due to the yellow colorant and the purple colorant contained. Therefore, unfortunately, this composition has low transmittance for light in the visible region.

In the case of the ultraviolet absorber into which a polymerizable group is introduced as described in JP2000-123621A and WO2019/073869A, bleed out and the like of the ultraviolet absorber from the resin composition can be suppressed to some extent. However, the ultraviolet absorber has absorption wavelength in a wavelength region shorter than ultraviolet light, and has low a absorbance at around a wavelength of 400 nm. Therefore, the ultraviolet absorber has poor blue light cut properties. In order to achieve sufficient cut properties, a large amount of the ultraviolet absorber needs to be added. Therefore, from the viewpoint of degree of freedom of formulating a composition, the ultraviolet absorber is still problematic for practical use.

An object to be achieved by an embodiment of the present invention is to provide a polymerizable composition and a resin composition that excellently cut off a long wavelength region of ultraviolet light and a short wavelength region of visible light and can form a cured substance inhibited from undergoing bleed out, outflow, precipitation, and the like of a compound, an ultraviolet cut film that contains the cured substance of the polymerizable composition or the resin composition, and a laminate that comprises the ultraviolet cut film.

An object to be achieved by another embodiment of the present invention is to provide a compound and a polymer that excellently cuts off a long wavelength region of ultraviolet light and a short wavelength region of visible light.

Means for achieving the above objects include the following aspects.
<1> A polymerizable composition containing a compound represented by General Formula (I-1) and a polymerizable compound.

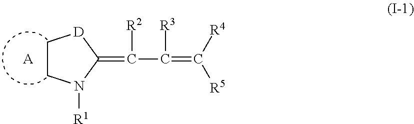

In General Formula (I-1), R¹ represents a hydrogen atom, an alkyl group, or an aryl group, R² and R³ each independently represent a hydrogen atom, an alkyl group, an aryl group, or a cyano group, and R⁴ and R⁵ each independently represent an electron-withdrawing group.

D represents an oxygen atom, a sulfur atom, or N-E, and E represents an alkyl group. A represents a 5- or 6-membered saturated or unsaturated ring, and the 5- or 6-membered ring may be further condensed.
<2> A polymerizable composition containing a compound represented by General Formula (I-2) and a polymerizable compound.

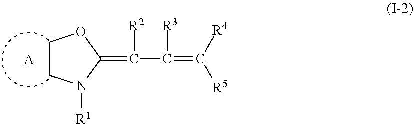

In General Formula (I-2), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, and the 5- or 6-membered ring may be further condensed.
<3> The polymerizable composition described in in <2>, in which R⁴ and R⁵ in General Formula (I-2) each independently represent a cyano group, an alkylsulfonyl group, an arylsulfonyl group, an alkoxycarbonyl group, a carbamoyl group, an alkylcarbonyl group, or an arylcarbonyl group.
<4> The polymerizable composition described in <2> or <3>, in which A in General Formula (I-2) represents a benzene ring or a naphthalene ring.
<5> The polymerizable composition described in any one of <2> to <4>, in which the compound represented by General Formula (I-2) has absorption maximum in a wavelength range of 390 nm to 430 nm in ethyl acetate.
<6> The polymerizable composition described in any one of <1> to <5> further containing an ultraviolet absorber other than the compound represented by General Formula (I-1) or the compound represented by General Formula (I-2).
<7> An ultraviolet cut film which is a cured substance of the polymerizable composition described in any one of <1> to <6>.
<8> A laminate having a support and the ultraviolet cut film described in <7>.
<9> A compound represented by General Formula (II).

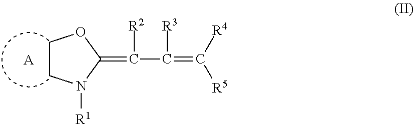

In General Formula (II), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, and the 5- or 6-membered ring may be further condensed.

Here, at least one of R¹, R², R³, R⁴, R⁵, or A contains a substituent selected from the group consisting of General Formula (III) and General Formula (IV), and in a case where at least one of R¹, R², R³, R⁴, or R⁵ contains a substituent selected from the group consisting of General Formula (III) and General Formula (IV), at least one of R¹, R², R³, R⁴, or R⁵ may be a substituent selected from the group consisting of General Formula (III) and General Formula (IV).

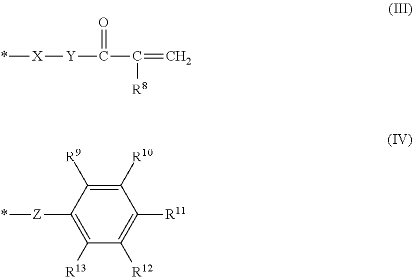

In General Formula (III), X represents a single bond or an alkylene group, Y represents a single bond, —O—, or —NR¹⁴—, and R¹⁴ represents a hydrogen atom or an alkyl group. R⁸ represents a hydrogen atom or an alkyl group. * Represents a binding position.

In General Formula (IV), R⁹, R¹⁰, R¹¹, R¹², and R¹³ each independently represent a hydrogen atom, a halogen atom, an alkyl group, or an alkoxy group, and Z represents a single bond or an alkylene group. * Represents a binding position. Here, at least one of R⁹, R¹⁰, R¹¹, R¹², or R¹³ represents a vinyl group.
<10> The compound described in <9>, in which R⁴ and R⁵ in General Formula (II) each independently represent a cyano group, an alkylsulfonyl group, an arylsulfonyl group, an alkoxycarbonyl group, a carbamoyl group, an alkylcarbonyl group, or an arylcarbonyl group.
<11> The compound described in <9> or <10>, in which A in General Formula (II) represents a benzene ring or a naphthalene ring.
<12> The compound described in any one of <9> to <11> that has absorption maximum in a wavelength range of 390 nm to 430 nm in ethyl acetate.
<13> A polymer containing a constitutional unit derived from the compound described in any one of <9> to <12>.
<14> A polymerizable composition containing the compound described in any one of <9> to <12>.
<15> The polymerizable composition described in <14>, further containing an ultraviolet absorber other than the compound represented by General Formula (II).
<16> A resin composition containing the compound described in any one of <9> to <12> and a polymer compound.
<17> A resin composition containing the polymer described in <13>.
<18> The resin composition described in <16> or <17>, further containing an ultraviolet absorber other than the compound represented by General Formula (II).
<19> An ultraviolet cut film that is a cured substance of the polymerizable composition described in <14> or <15> or a cured substance of the resin composition described in any one of <16> to <18>.
<20> A laminate having a support and the ultraviolet cut film described in <19>.

According to an embodiment of the present invention, it is possible to provide a polymerizable composition and a resin composition that excellently cut off a long wavelength region of ultraviolet light and a short wavelength region of visible light and can form a cured substance inhibited from undergoing bleed out, outflow, precipitation, and the like of a compound, an ultraviolet cut film that contains the cured substance of the polymerizable composition or the resin composition, and a laminate that comprises the ultraviolet cut film.

According to another embodiment of the present invention, it is possible to provide a compound and a polymer that excellently cuts off a long wavelength region of ultraviolet light and a short wavelength region of visible light.

### BRIEF DESCRIPTION OF THE DRAWINGS

FIG. 1 is a graph showing the transmittance of an ultraviolet cut film 1 at a wavelength of 300 nm to 800 nm, the ultraviolet cut film 1 being a cured substance of a polymerizable composition of Example 7.

FIG. 2 is a graph showing the transmittance of an ultraviolet cut film 2 at a wavelength of 300 nm to 800 nm, the ultraviolet cut film 2 being a cured substance of a polymerizable composition of Example 8.

FIG. 3 is a graph showing an absorption spectrum that an exemplary polymer B obtained in Example 10 has in a chloroform solution.

### DESCRIPTION OF THE PREFERRED EMBODIMENTS

Hereinafter, the polymerizable composition, compound, resin composition, ultraviolet cut film, and laminate according to the present disclosure will be specifically described with reference to specific examples.

However, the present disclosure is not at all limited to the following embodiments. Within the scope of the object of the present disclosure, the present disclosure can be embodied by being appropriately modified.

In the present disclosure, a numerical range described using “to” means a range including numerical values described before and after “to” as a minimum value and a maximum value, respectively.

Regarding the numerical ranges described in stages in the present disclosure, the upper or lower limit of a numerical range may be replaced with the upper or lower limit of another numerical range described in stages. Furthermore, regarding the numerical ranges described in the present disclosure, the upper or lower limit of a numerical range may be replaced with values described in examples.

In addition, in the present disclosure, the combination of two or more preferred aspects is a more preferred aspect.

In the present disclosure, in a case where there are two or more kinds of substances corresponding to each component, unless otherwise specified, the content of each component means the total content or total mixing ratio of two or more kinds of substances.

In the present disclosure, “(meth)acryloyl” means at least one of acryloyl or methacryloyl, and “(meth)acrylate” means at least one of acrylate or methacrylate.

Unless otherwise specified, “substituent” means both the unsubstituted substituent and substituent further having a substituent. For example, “alkyl group” means both the unsubstituted alkyl group and alkyl group further having a substituent. The same shall be applied to other substituents.

In the present disclosure, unless otherwise specified, the description of the number of carbon atoms in a substituent, such as “alkyl group having 1 to 3 carbon atoms”, means the number of carbon atoms in a molecule constituting a substituent such as an unsubstituted alkyl group, and does not mean the total number of carbon atoms in a substituent that further has other substituents. The same shall be applied to the number of carbon atoms in a substituent other than an alkyl group.

In the present disclosure, the term “step” includes not only an independent step, but also a step that is not clearly distinguished from other steps as long as the step achieves the intended goal.

In the present disclosure, “ultraviolet rays” means not only the ultraviolet wavelength range of 10 nm to 400 nm that is shorter than the visible light and longer than the soft X-rays, but also the wavelength region of light in a long wavelength region of ultraviolet light and the light in a short wavelength region of visible light.

The light in a long wavelength region of ultraviolet light and the light in a short wavelength region of visible light mean, for example, at least a wavelength range of 390 nm to 430 nm.

“Ultraviolet cut” means not only a case where the ultraviolet rays are totally cut off through a cured substance of a polymerizable composition or a cured substance of a resin composition, but also a case where at least some of the ultraviolet rays are cut off and the ultraviolet transmittance of the cured substance is reduced.

In the present disclosure, a phenomenon where a certain compound is released from a cured substance, that is, a phenomenon including at least one of bleed out, precipitation, or elution will be simply described as “bleed out and the like” in some cases.

[Polymerizable Composition]

A first aspect of the polymerizable composition of the present disclosure contains a compound represented by General Formula (I-1) and a polymerizable compound.

Hereinafter, the compound represented by General Formula (I-1) will be called “specific compound (I-1)” in some cases. In addition, the first aspect of the polymerizable composition containing the specific compound (I-1) and the polymerizable compound will be called “polymerizable composition (I-1)”.

[Specific Compound (I-1)]

The specific compound (I-1) contained in the polymerizable composition (I-1) of the present disclosure is a compound represented by General Formula (I-1).

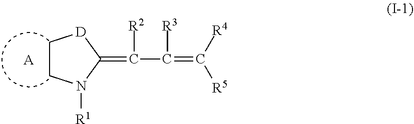

In General Formula (I-1), R¹ represents a hydrogen atom, an alkyl group, or an aryl group, R² and R³ each independently represent a hydrogen atom, an alkyl group, an aryl group, or a cyano group, and R⁴ and R⁵ each independently represent an electron-withdrawing group.

D represents an oxygen atom, a sulfur atom, or N-E, and E represents an alkyl group. A represents a 5- or 6-membered saturated or unsaturated ring, and the 5- or 6-membered ring may be further condensed.

In General Formula (I-1), D represents an oxygen atom, a sulfur atom, or N-E, and E represents an alkyl group. As the alkyl group represented by E, an alkyl group having 1 to 5 carbon atoms is preferable, and a methyl group, an ethyl group, or a propyl group is more preferable.

As D, an oxygen atom or a sulfur atom is preferable, and an oxygen atom is more preferable.

Examples of more preferable aspects of the compound represented by General Formula (I-1) include a compound represented by General Formula (I-2) that will be described later, in which D in General Formula (I-1) represents an oxygen atom.

R¹, R², R³, R⁴, R⁵, and A that represent substituents or partial structures in General Formula (I-1) are the same as the substituents or partial structures in General Formula (I-2) that will be described later, and preferable aspects thereof are also the same. Therefore, only the substituents and partial structures of General Formula (I-2) will be specifically described, and the substituents and partial structures in General Formula (I-1) will not be described.

[Polymerizable Composition]

A second aspect of the polymerizable composition of the present disclosure contains a compound represented by General Formula (I-2) and a polymerizable compound.

The polymerizable composition of the present disclosure is a polymerizable composition containing a compound represented by General Formula (I-2), which is a preferable aspect of the compound represented by General Formula (I-1) described above.

Hereinafter, the compound represented by General Formula (I-2) will be called “specific compound (I-2)” in some cases. In addition, the second aspect of the polymerizable composition containing the specific compound (I-2) and a polymerizable compound will be called “polymerizable composition (I-2)”.

Hereinafter, the compound represented by General Formula (I-1) and the compound represented by General Formula (I-2) that is a preferable aspect of the compound represented by General Formula (I-1) will be specifically described.

[Specific Compound (I-2)]

The specific compound (I-2) contained in the polymerizable composition (I-2) of the present disclosure is a compound represented by General Formula (I-2) and has a merocyanine skeleton.

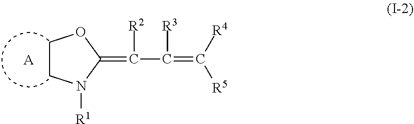

In General Formula (I-2), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, and the 5- or 6-membered ring may be further condensed.

In General Formula (I-2), R¹ represents a hydrogen atom, an alkyl group, or an aryl group.

In a case where R¹ represents an alkyl group, the alkyl group is preferably an alkyl group having 1 to 20 carbon atoms.

The alkyl group may be linear or branched, or may have a ring structure. Furthermore, the alkyl group may be unsubstituted or may have a substituent.

In a case where the alkyl group has a substituent, examples of substituents that can be introduced into the alkyl group include a halogen atom, an aryl group, an alkoxy group, an alkoxycarbonyl group, an acyloxy group, an amide group, a carbamoyl group, an aryloxy group, an alkylthio group, an arylthio group, an alkylsulfonyl group, and the like.

Preferable examples of the alkyl group represented by R¹ include methyl, ethyl, propyl, butyl, amyl, isoamyl, hexyl, octyl, decyl, dodecyl, benzyl, (meth)acryloyloxyethyl, 1-(meth)acryloyloxypropane-2-yl, 2-(meth)acryloyloxypropan-1-yl, (meth)acryloyloxybutyl, (meth)acryloyloxyhexyl, (meth)acryloyloxyoctyl, 4-vinylbenzyl, 3-vinylbenzyl, 2-vinylbenzyl, and the like.

In a case where R¹ represents an aryl group, the aryl group is preferably an aryl group having 6 to 20 carbon atoms.

As the aryl group represented by R¹, a phenyl group, a 1-naphthyl group, a 2-naphthyl group, and the like are preferable.

In a case where the aryl group has a substituent, examples of substituents that can be introduced into the aryl group include a halogen atom, an aryl group, an alkoxy group, an alkoxycarbonyl group, an acyloxy group, an amide group, a carbamoyl group, an aryloxy group, an alkylthio group, an arylthio group, an alkylsulfonyl group, and the like.

Particularly, R¹ is preferably an alkyl group or an aryl group, and more preferably an unsubstituted alkyl group having 1 to 5 carbon atoms, an alkyl group or an aryl group having a double bond as a substituent at the terminal, and the like. From the viewpoint of making it easy for a gram absorption coefficient of the compound to fall into a suitable range, le is even more preferably an unsubstituted alkyl group having 1 to 5 carbon atoms.

In General Formula (I-2), R² and R³ each independently represent a hydrogen atom, an alkyl group, an aryl group, or a cyano group.

In a case where R² and R³ each represent an alkyl group, the alkyl group is preferably an alkyl group having 1 to 20 carbon atoms, and more preferably methyl or ethyl.

In a case where R² and R³ each represent an aryl group, the aryl group is preferably an aryl group having 6 to 20 carbon atoms, and more preferably phenyl, 1-naphthyl, or 2-naphthyl.

Especially, from the viewpoint of making it easy for the gram absorption coefficient of the compound to fall into a suitable range and from the viewpoint of ease of manufacturing, it is more preferable that R² and R³ both represent a hydrogen atom.

In General Formula (I-2), R⁴ and R⁵ each independently represent an electron-withdrawing group.

The electron-withdrawing group in the present disclosure refers to a substituent having a positive σₚ Hammett constant. Details of σₚ Hammett constant are described in Hansch, C.; Leo, A.; Taft, R. W. Chem. Rev. 1991, 91, 165-195.

Specifically, as the electron-withdrawing group represented by R⁴ and R⁵, a cyano group, an alkylsulfonyl group, an arylsulfonyl group, an alkoxycarbonyl group, a carbamoyl group, an alkylcarbonyl group, an arylcarbonyl group, and the like are preferable from the view point of easily obtaining a compound having suitably absorption wavelength and absorption waveform, and a cyano group, an alkylsulfonyl group, an arylsulfonyl group, an alkoxycarbonyl group, an alkylcarbonyl group, and the like are more preferable.

Specific examples of the alkylsulfonyl group include methylsulfonyl, ethylsulfonyl, propyl sulfonyl, butyl sulfonyl, hexylsulfonyl, octylsulfonyl, decylsulfonyl, dodecyl sulfonyl, benzyl sulfonyl, 4-chlorobenzylsulfonyl, 4-methoxybenzylsulfonyl, 4-vinylbenzylsulfonyl, and the like.

Specific examples of the arylsulfonyl group include phenylsulfonyl, 1-naphthyl sulfonyl, 4-methylphenylsulfonyl, 4-chlorophenylsulfonyl, 4-methoxyphenylsulfonyl, 4-phenylsulfonyl, and the like.

Specific examples of the alkoxycarbonyl group include methoxycarbonyl, ethoxycarbonyl, butoxycarbonyl, 2-(meth)acryloyloxyethoxycarbonyl, 3-(meth)acryloyloxy-2-hydroxypropoxycarbonyl, 4-(meth)acryloyloxybutoxycarbonyl, 6-(meth)acryloyloxyhexyloxycarbonyl, 8-(meth)acryloyloxyoctyloxycarbonyl, 1-(meth)acryloyloxy-2-propyloxycarbonyl, 2-(meth)acryloyloxypropan-1-yloxycarbonyl, 4-vinylbenzyloxycarbonyl, 3-vinylbenzyloxycarbonyl, and the like.

Specific examples of the carbamoyl group include unsubstituted carbamoyl, N-methylcarbamoyl, N-ethylcarbamoyl, N,N-dimethylcarbamoyl, morpholinocarbonyl, pyrrolidinocarbonyl, piperidinocarbonyl, and the like.

Specific examples of the alkylcarbonyl group include acetyl, propanoyl, butanoyl, dimethylacetyl, pivaloyl, acryloyl, methacryloyl, and the like.

Specific examples of arylcarbonyl include benzoyl, 4-methoxybenzoyl, 4-methylbenzoyl, thenoyl, and the like.

R⁴ and R⁵ in General Formula (I-2) may form a ring by being bonded to each other, or may not form a ring. From the viewpoint of further improving absorptivity for a short wavelength region of visible light and obtaining a sharper absorption peak, it is preferable that R⁴ and R⁵ be not bonded to each other to form a ring.

In General Formula (I-2), A represents a 5- or 6-membered saturated or unsaturated ring, and the 5- or 6-membered ring may be further condensed. Particularly, from the viewpoint of easily obtaining a compound having suitable absorption wavelength and suitable absorption waveform, A is preferably a 6-membered unsaturated ring.

Specific examples of A include a cyclohexane ring, a cyclohexene ring, a cyclohexadiene ring, a benzene ring, a tetrahydronaphthalene ring, a naphthalene ring, and the like. The ring represented by A may be substituted with a halogen atom, an alkyl group, an aryl group, an alkoxy group, an aryloxy group, an alkoxycarbonyl group, a cyano group, or the like.

As A, from the viewpoint of easily obtaining a compound having suitable absorption wavelength and suitable absorption waveform, a benzene ring or a naphthalene ring is preferable, and an unsubstituted benzene ring, an unsubstituted naphthalene ring, or benzene having an alkyl group, an alkoxy group, or a cyano group as a substituent is more preferable.

It is preferable that the compound represented by General Formula (I-1) and the compound represented by General Formula (I-2) have absorption maximum in a wavelength range of 390 nm to 430 nm in ethyl acetate.

The maximal absorption wavelength of the specific compound (I-1) and the specific compound (I-2) can be measured using, for example, a spectrophotometer.

More specifically, a spectroscopic spectrum of a 0.005% by mass solution of the specific compound (I-1) or the specific compound (I-2) prepared by dissolving the specific compound (I-1) or the specific compound (I-2) in ethyl acetate can be measured at room temperature (25° C.) by using a 1 cm quartz cell.

In the present disclosure, the maximal absorption wavelength of a compound refers to the maximal absorption wavelength max in a spectroscopic spectrum measured using a ultraviolet/visible spectrophotometer UV-1800 (trade name) manufactured by Shimadzu Corporation.

The specific compound (I-1) or the specific compound (I-2) has high solubility in an organic solvent and/or the polymerizable compound (polymerizable monomer) that will be described later. More specifically, the specific compound (I-1) or (I-2) dissolves 0.1% by mass or more in ethyl acetate at 25° C., for example.

The specific compound (I-1) and the specific compound (I-2) can be obtained with reference to the compounds described in the following documents and the like and compounds similar to these.

Justus Liebigs Annalen der Chemie (1954), 587, p 195-206, DE 10203939 A1, JP1993-100351A (JP-05-100351A), EP297871A, EP297872A, Helvetica Chimica Acta (1987) 70(6), p 1583-1595, U.S. Pat. No. 4,283,487A, Justus Liebigs Annalen der Chemie (1971), 749, p 183-9′7, U.S. Pat. No. 3,244,526A, JP1993-100348A (JP-05-100348A), JP1993-093980A (JP-05-093980A), JP1993-011383A (JP-05-011383A), JP1997-291220A (JP-09-291220A).

Specific examples of the specific compound (I-1) or the specific compound (I-2) will be shown below. The present disclosure is not limited to the following specific examples.

Examples of an exemplary compound that is the compound represented by the specific compound (I-1) and is not included in the compound represented by the specific compound (I-2) include the following exemplary compound I-1-1 to exemplary compound I-1-16.

Examples of an exemplary compound that is the compound represented by the specific compound (I-2) and does not have a double bond in the molecule include the following exemplary compound I-2-1 to exemplary compound I-2-18.

In the following exemplary compound I-1-1 to exemplary compound I-2-18, Me represents a methyl group, and Et represents an ethyl group.

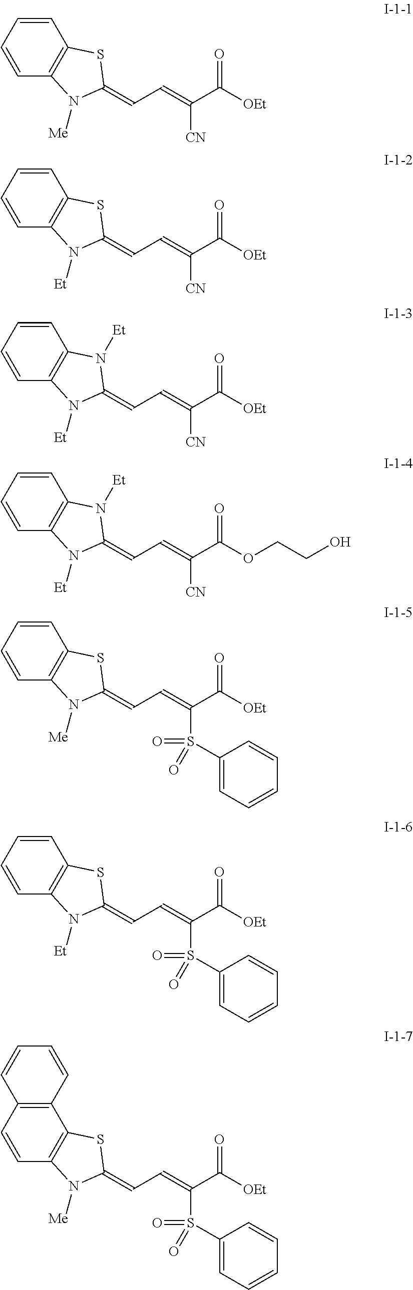

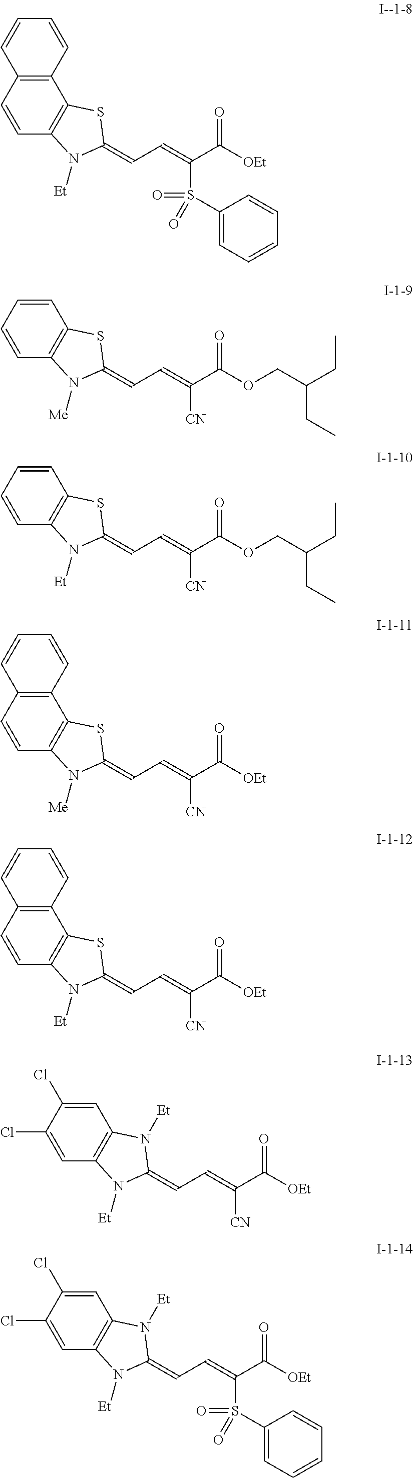

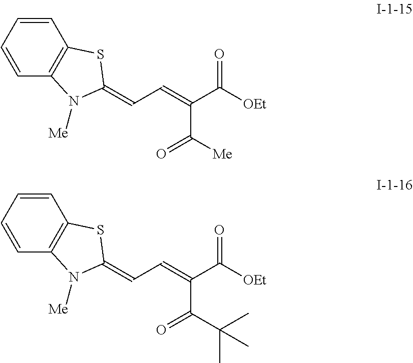 [SMILES: CCOC(=O)/C(=C/C=C1\Sc2ccccc2N1C)C(C)=O ; CCOC(=O)/C(=C/C=C1\Sc2ccccc2N1C)C(=O)C(C)(C)C]

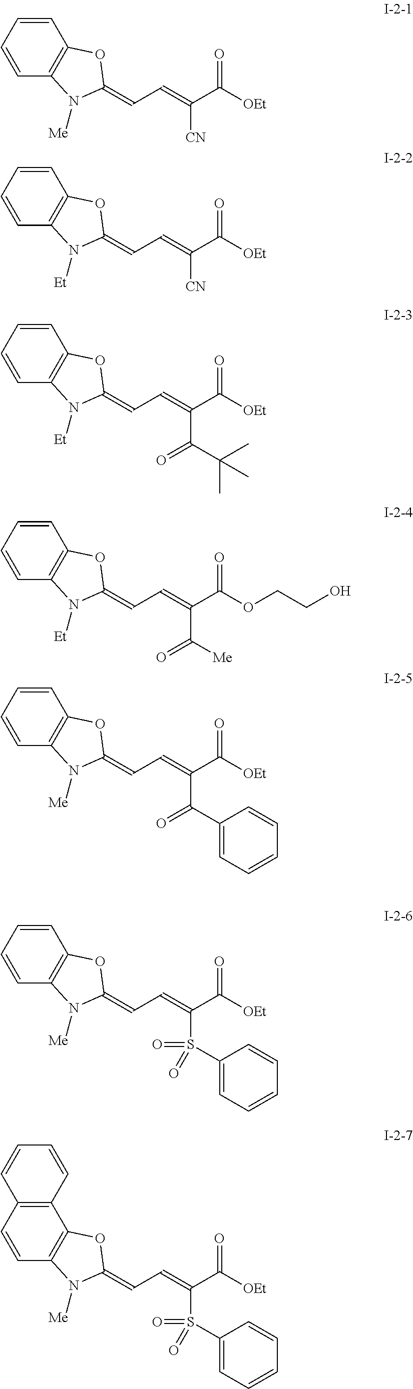

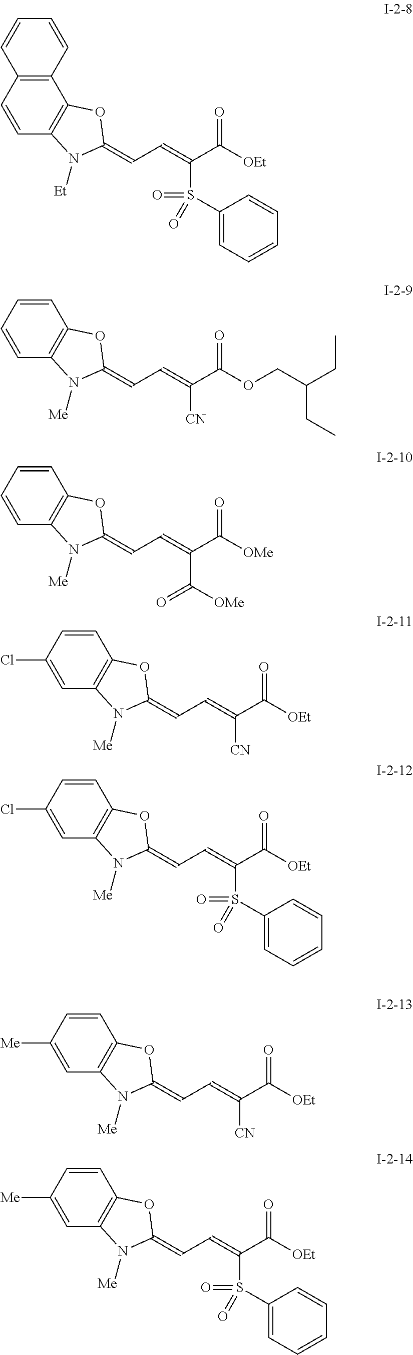

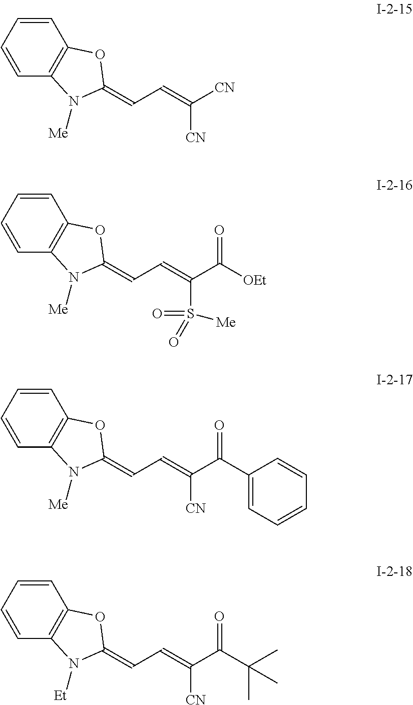 [SMILES: CN1/C(=C/C=C(C#N)C#N)Oc2ccccc21 ; CCOC(=O)/C(=C/C=C1\Oc2ccccc2N1C)S(C)(=O)=O ; CN1/C(=C/C=C(\C#N)C(=O)c2ccccc2)Oc2ccccc21 ; CCN1/C(=C/C=C(\C#N)C(=O)C(C)(C)C)Oc2ccccc21]

Examples of the exemplary compound as the specific compound (I-2) having a double bond in the molecule include an exemplary compound 1 to an exemplary compound 62 listed as specific examples of the compound represented by General Formula (II) that will be described later.

The compound represented by the specific compound (I-1) and the compound represented by General Formula (I-2) absorb light which is a long wavelength region of ultraviolet rays and a short wavelength region of visible light. More specifically, the specific compound (I-1) and the specific compound (I-2) efficiently absorb light having a wavelength of around 390 nm to 430 nm. Furthermore, the specific compound (I-1) and the specific compound (I-2) are extremely minimally absorb the long wavelength region of visible light and are less colored. From such a viewpoint, it is preferable that the specific compound (I-1) and the specific compound (I-2) have a sharp peak of maximal absorption wavelength.

The light absorption coefficient of the specific compound (I-1) and the specific compound (I-2) is preferably 20,000 or more, more preferably 30,000 or more, and particularly preferably 40,000 or more.

A ratio [ε(440)/ε(405)] of a light absorption coefficient [ε(440)] of the specific compounds (I-1) and (I-2) at 440 nm to a light absorption coefficient [4405)] of the specific compounds (I-1) and (I-2) at 405 nm is preferably 0.05 or less, more preferably 0.025 or less, and particularly preferably 0.0125 or less.

A compound having a low [ε(440)/ε(405)] ratio is preferable because this compound efficiently cuts off light which is the long wavelength region of ultraviolet rays and the short wavelength region of visible light (that is, blue light) and is extremely minimally colored.

The polymerizable composition (I-1) may contain only one kind of specific compound (I-1) or two or more kinds of specific compounds (I-1).

The polymerizable composition (I-2) may contain only one kind of specific compound (I-2) or two or more kinds of specific compounds (I-2).

The content of the specific compound (I-1) in the polymerizable composition (I-1) and the content of the specific compound (I-2) in the polymerizable composition (I-2) are not particularly limited, and can be appropriately selected depending on the purpose.

From the viewpoint of excellent balance between the ultraviolet cut effect and the visibility through a cured substance in a case where the polymerizable composition (I-1) or the polymerizable composition (I-2) is made into a cured substance, the content of the specific compound (I-1) or the specific compound (I-2) in the polymerizable composition (I-1) or the polymerizable composition (I-2) is preferably in a range of 0.005 mmol (millimoles)/m² to 0.1 mmol/m², and more preferably in a range of 0.01 mmol/m² to 0.05 mmol/m².

In addition, from the viewpoint of excellent balance between the ultraviolet cut effect and the visibility through a cured substance in a case where the polymerizable composition (I-1) or the polymerizable composition (I-2) is made into a cured substance, the content of the specific compound (I-1) or the specific compound (I-2) with respect to the total solid content of the polymerizable composition (I-1) or the polymerizable composition (I-2) is preferably 0.01% by mass to 5% by mass, more preferably 0.1% by mass to 3% by mass, and even more preferably 0.3% by mass to 2% by mass.

“Total solid content” refers to the total amount of components of the composition, except for solvents. Some of the components are liquid components such as a sort of low-molecular-weight monomers, but liquid components other than solvents are also included in the solid content in the present disclosure.

The polymerizable composition (I-1) or the polymerizable composition (I-2) of the present disclosure contains the polymerizable compound that will be described later. In a case where the polymerizable composition (I-1) or (I-2) is formed into a cured substance by a polymerization reaction, the specific compound (I-1) or the specific compound (I-2) in the cured substance is immobilized. Therefore, from the polymerizable composition (I-1) or the polymerizable composition (I-2), a cured substance can be obtained in which bleed out, outflow, precipitation, and the like of the specific compound (I-1) or the specific compound (I-2) are suppressed.

The specific compound (I-1) or the specific compound (I-2) contained in the polymerizable composition (I-1) or the polymerizable composition (I-2) may or may not have a polymerizable group in the molecule. However, it is preferable that the specific compound (I-1) or (I-2) have a polymerizable group because then the bleed out and the like are more markedly suppressed.

[Polymerizable Compound]

The polymerizable composition (I-1) or the polymerizable composition (I-2) of the present disclosure contains a polymerizable compound.

The polymerizable compound is not particularly limited as long as it is a compound that can be polymerized and cured by the application of energy. Examples of the polymerizable compound include a polymerizable compound having at least one ethylenically unsaturated double bond.

The polymerizable compound in the present disclosure is preferably selected from the group consisting of a compound having one terminal ethylenically unsaturated bond and a compound having two or more terminal ethylenically unsaturated bonds. The group of compounds having a terminal ethylenically unsaturated bond is widely known in the field of related industries. In the polymerizable composition (I-1) or the polymerizable composition (I-2) of the present disclosure, known polymerizable compounds can be used without particular limitation.

The polymerizable compound can take chemical forms, for example, a monomer, a prepolymer such as a dimer, a trimer, or an oligomer, a mixture and a (co)polymer of these, and the like.

Examples of the monomer and the (co)polymer thereof include unsaturated carboxylic acids (such as acrylic acid, methacrylic acid, itaconic acid, crotonic acid, isocrotonic acid, and maleic acid), esters and amides thereof, and (co) polymers of the aforementioned components.

Specific preferable examples thereof include a (meth)acrylate monomer.

Examples of the (meth)acrylate monomer include methyl (meth)acrylate, ethyl (meth)acrylate, butyl (meth)acrylate, 2-hydroxyethyl (meth)acrylate, glycidyl (meth)acrylate, benzyl (meth) acrylate, 2-(2-phenoxy)ethyl (meth)acrylate, 2-ethylhexyl (meth)acrylate, n-octyl (meth)acrylate, isooctyl (meth)acrylate, n-nonyl (meth)acrylate, isononyl (meth)acrylate, n-decyl (meth)acrylate, isodecyl (meth)acrylate, n-dodecyl (meth)acrylate, n-tridecyl (meth)acrylate, n-tetradecyl (meth)acrylate, n-hexadecyl (meth)acrylate, stearyl (meta)acrylate, isobornyl (meth)acrylate, dicyclopentenyl (meth)acrylate, dicyclopentanyl (meth)acrylate, dicyclopentenyloxyethyl (meth)acrylate, 1-hydroxyheptyl (meth)acrylate, 1-hydroxybutyl (meth)acrylate, 1-hydroxypentyl (meth)acrylate, 2-hydroxybutyl (meth)acrylate, ethylene glycol diacrylate, ethylene glycol dimethacrylate, polyethylene glycol diacrylate, polyethylene glycol dimethacrylate, ethoxylated bisphenol A diacrylate, ethoxylated bisphenol A dimethacrylate, ethoxylated trimethylolpropane triacrylate, ethoxylated trimethylolpropane trimethacrylate, ethoxylated glycerin triacrylate, ethoxylated glycerin trimethacrylate, ethoxylated pentaerythritol tetraacrylate, ethoxylated pentaerythritol tetramethacrylate, ethoxylated dipentaerythritol hexaacrylate, polyglycerin monoethylene oxide polyacrylate, polyglycerin polyethylene glycol polyacrylate, dipentaerythritol hexaacrylate, dipentaerythritol hexamethacrylate, neopentyl glycol diacrylate, neopentyl glycol dimethacrylate, pentaerythritol triacrylate, pentaerythritol trimethacrylate, trimethylolpropane triacrylate, trimethylolpropane trimethacrylate, tricyclodecanedimethanol diacrylate, tricyclodecanedimethanol dimethacrylate, 1,6-hexanediol diacrylate, 1,6-hexanediol dimethacrylate, and the like.

The specific preferable examples also include a styrene monomer.

Examples of the styrene monomer include styrene, methylstyrene, dimethylstyrene, trimethylstyrene, ethylstyrene, fluorostyrene, chlorostyrene, methoxystyrene, t-butoxystyrene, divinylbenzene, and the like.

Details of how to use the polymerizable compound, such as the structure of the polymerizable compound, whether one kind of polymerizable compound will be used alone or two or more kinds of polymerizable compounds will be used in combination, and the content of the polymerizable compound, can be arbitrarily set in consideration of the design of ultimate performance of the polymerizable composition (I-1) or the polymerizable composition (I-2).

For example, from the viewpoint of sensitivity, it is preferable that the polymerizable compound have a structure containing large amounts of unsaturated groups per molecule. In many cases, it is preferable that the polymerizable compound have two or more functional groups. Furthermore, from the viewpoint of increasing the strength of the obtained cured substance, for example, the ultraviolet cut film, it is possible to use a compound having three or more functional groups, for example, a hexafunctional acrylate compound or the like.

Furthermore, as a method for adjusting both the sensitivity and strength, it is also effective use compounds having different numbers of functional groups or having different polymerizable groups, for example, an acrylic acid ester, a methacrylic acid ester, a styrene-based compound, a vinyl ether-based compound, and the like in combination.

The polymerizable composition (I-1) or the polymerizable composition (I-2) may contain only one kind of polymerizable compound, or two or more kinds of polymerizable compounds may be used in combination.

The content of the polymerizable compound in the polymerizable composition (I-1) or the polymerizable composition (I-2) is not particularly limited.

For example, the content of the polymerizable compound in the total solid content of the polymerizable composition (I-1) or the polymerizable composition (I-2) can be 30% by mass or more and less than 100% by mass, and is preferably 50% by mass or more and less than 100% by mass and more preferably 60% by mass or more and less than 100% by mass. Considering the correlation between the polymerizable compound and other components contained in the polymerizable composition, the upper limit of the content of the polymerizable compound can be, for example, 99.99% by mass, 99.9% by mass, or the like.

As long as the effects of the polymerizable composition (I-1) or the polymerizable composition (I-2) of the present disclosure are not impaired, the polymerizable composition (I-1) or (I-2) may further contain optional components that can be used in the polymerizable composition, in addition to the specific compound (I-1), the specific compound (I-2), and a polymerizable compound. Examples of the optional components (hereinafter, called other components) include a polymerization initiator, a surfactant, other ultraviolet absorbers different from the specific compound (I-1) or the specific compound (I-2), a colorant, and the like.

(Polymerization Initiator)

The polymerizable composition (I-1) or the polymerizable composition (I-2) of the present disclosure may contain a polymerization initiator.

The polymerization initiator is not particularly limited as long as it is a compound capable of generating an initiating species necessary for polymerization by the application of energy. The polymerization initiator to be used can be appropriately selected from known photopolymerization initiators and thermal polymerization initiators.

As a photopolymerization initiator, for example, a compound having photosensitivity to rays that span the ultraviolet region and the visible region is preferable. Furthermore, the photopolymerization initiator may be an activator that causes a certain action with a photoexcited sensitizer and generates active radicals. A photopolymerization initiator that generates active radicals by light is sometimes called a photoradical polymerization initiator.

As the photoradical polymerization initiator, known photoradical polymerization initiators can be used without particular limitation. Examples of the photoradical polymerization initiator include a halogenated hydrocarbon derivative such as a photopolymerization initiator having a triazine skeleton or a photopolymerization initiator having an oxadiazole skeleton, an acylphosphine oxide compound, hexaarylbiimidazole, an oxime derivative, an aminoacetophenone compound, a hydroxyacetophenone compound, and the like. Specifically, examples thereof include acylphosphine oxide such as 2,4,6-trimethylbenzoyldiphenylphosphine oxide and bis(2,4,6-trimethylbenzoyl)phenylphosphine oxide, an oxime ester-based compound such as Irgacure OXE01, Irgacure OXE02, and Irgacure OXE03 (all manufactured by BASF SE), α-hydroxyacetophenone such as Omnirad (former name: Irgacure) 1173, Omnirad (former name: Irgacure) 2959, and Omnirad (former name: Irgacure) 127 (all manufactured by BASF SE), α-aminoacetophenone SUCH AS Omnirad (former name: Irgacure) 907 and Omnirad (former name: Irgacure) 369 (all manufactured by BASF SE), and the like.

In a case where the polymerizable composition (I-1) or the polymerizable composition (I-2) contains a photoradical polymerization initiator, the content of the photoradical polymerization initiator is not particularly limited. Generally, for example, the content of the photoradical polymerization initiator with respect to the total solid content of the polymerizable composition (I-1) or the polymerizable composition (I-2) can be 0.1% by mass to 20% by mass, and is preferably 0.3% by mass to 15% by mass and more preferably 0.4% by mass to 10% by mass.

A thermal polymerization initiator that generates active radicals by heating is sometimes called a thermal radical polymerization initiator.

As the thermal radical polymerization initiator, known thermal radical polymerization initiators can be used without particular limitation. Examples of the thermal radical polymerization initiator include azo-based compounds such as 2,2′-azobisisobutyronitrile, 2,2′-azobis(2,4-dimethyl-4-methoxyvaleronitrile), 2,2′-azobis(2,4-dimethylvaleronitrile), dimethyl-2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl 1,1′-azobis(1-cyclohexanecarboxylate), and 2,2′-azobis[2-(2-imidazolin-2-yl)propane] dihydrochloride; organic peroxides such as 1,1-di(t-hexylperoxy)cyclohexane, 1,1-di(t-butylperoxy)cyclohexane, 2,2-di(4,4-di-(t-butylperoxy)cyclohexyl)propane, t-hexylperoxyisopropyl monocarbonate, t-butylperoxy-3,5,5-trimethylhexanoate, t-butylperoxylaurate, dicumyl peroxide, di-t-butyl peroxide, t-butylperoxy-2-ethylhexanoate, t-hexylperoxy-2-ethylhexanoate, cumene hydroperoxide, and t-butylhydroperoxide; inorganic peroxides such as potassium persulfate, ammonium persulfate, and hydrogen peroxide; and the like.

In a case where the polymerizable composition (I-1) or the polymerizable composition (I-2) contains a thermal radical polymerization initiator, the content of the thermal radical polymerization initiator is not particularly limited. Generally, for example, the content of the thermal radical polymerization initiator with respect to the total solid content of the polymerizable composition (I-1) or the polymerizable composition (I-2) can be 0.1% by mass to 20% by mass, and is preferably 0.3% by mass to 15% by mass and more preferably 0.4% by mass to 10% by mass.

In a case where the polymerizable composition (I-1) or the polymerizable composition (I-2) contains a polymerization initiator, the polymerizable composition (I-1) or the polymerizable composition (I-2) may contain only one kind of polymerization initiator or may contain two or more kinds of polymerization initiators as necessary.

(Ultraviolet Absorber Other than Specific Compound)

The polymerizable composition (I-1) or the polymerizable composition (I-2) of the present disclosure may further contain an ultraviolet absorber other than the specific compound (I-1) or the specific compound (I-2).

The compound represented by General Formula (I-1) or the compound represented by General Formula (I-2) described above, that is, the specific compound (I-1) or the specific compound (I-2) preferably has absorption maximum in a wavelength range of 390 nm to 430 nm in ethyl acetate, and excellently absorbs, that is, excellent cuts off the long wavelength region of ultraviolet rays and short wavelength region of visible light.

In a case where the polymerizable composition (I-1) or the polymerizable composition (I-2) contains an ultraviolet absorber other than the specific compound (I-1) or the specific compound (I-2) (hereinafter, called other ultraviolet absorbers in some cases), particularly, other ultraviolet absorbers having absorption maximum in a wavelength region different from that absorbed into the specific compound (I-1) or the specific compound (I-2), it is possible to control the wavelength region of ultraviolet rays that the polymerizable composition can cut off.

It is preferable that the polymerizable composition (I-1) or (I-2) contain an ultraviolet absorber absorbing a shorter wavelength region compared to the specific compound (I-1) or the specific compound (I-2), because then the obtained polymerizable composition (I-1) or the polymerizable composition (I-2) excellently cuts off the wavelength of around 400 nm due to the specific compound (I-1) or the specific compound (I-2) and excellently absorbs ultraviolet rays having shorter wavelengths, and a cured substance of the polymerizable composition excellently cuts off ultraviolet rays in a broader wavelength region.

Examples of those other ultraviolet absorbers that can be contained in the polymerizable composition (I-1) or the polymerizable composition (I-2) of the present disclosure include an aminobutadiene-based compound, a dibenzoylmethane-based compound, a benzophenone-based compound, a benzotriazole-based compound, a hydroxyphenyltriazine-based compound, and the like. Among these, in view of higher compatibility with the specific compound (I-1) or the specific compound (I-2), a benzotriazole-based compound, a benzophenone-based compound, a hydroxyphenyltriazine-based compound, and the like are preferable.

“Benzotriazole-based compound” as the aforementioned other ultraviolet absorbers refers to a compound having at least “benzotriazole skeleton” in the molecule. Likewise, the name “X-based compound” for other compounds also refers to a compound having at least “skeleton X” in the molecule.

Those other ultraviolet absorbers that can be used in the present disclosure may or may not have a polymerizable group in the molecule.

As those other ultraviolet absorbers having a polymerizable group in the molecule, it is possible to use the compounds described in JP2003-129033A, JP2003-128730A, JP5518613B, JP2014-77076A, JP2015-168822A, JP2015-164994A, JP5868465B, JP2017-503905A, EP2951163B, WO2015/064674A, WO2015/064675A, EP2379512B, WO2017/102675A, JP6301526B, JP2017-503905A, JP5868465B, JP2018-135282A, JP2018-168089A, WO2019/087983A, such as a (2-(2-hydroxyphenyl) benzotriazole-based compound, a 2-hydroxybenzophenone-based compound, and a 2-(2-hydroxyphenyl)-1,3,5-triazine-based compound. Examples of commercially available products of these include 2-[2′-hydroxy-5′-(methacryloyloxyethyl)phenyl]-2H-benzotriazole (trade name “RUVA-93” manufactured by Otsuka Chemical Co., Ltd.).

In a case where the polymerizable composition (I-1) or the polymerizable composition (I-2) of the present disclosure contains other ultraviolet absorbers, the content of those other ultraviolet absorbers with respect to the total solid content of the polymerizable composition (I-1) or the polymerizable composition (I-2) is preferably 0.01% by mass to 10% by mass, and more preferably 0.01% by mass to 5% by mass.

In a case where the polymerizable composition (I-1) or the polymerizable composition (I-2) of the present disclosure contains other ultraviolet absorbers, the polymerizable composition (I-1) or (I-2) may contain only one kind of other ultraviolet absorbers or two or more kinds of other ultraviolet absorbers. In a case where the polymerizable composition contains two or more kinds of other ultraviolet absorbers, the content of the ultraviolet absorbers is preferably in the range described above.

(Solvent)

The polymerizable composition (I-1) or the polymerizable composition (I-2) can contain a solvent. In a case where the polymerizable composition (I-1) or or the polymerizable composition (I-2) composition (I-2) contains a solvent, the viscosity of the polymerizable composition (I-1) or (I-2) can be adjusted, and the prepared polymerizable composition (I-1) or polymerizable composition (I-2) can have viscosity suitable for the composition to be used as a coating liquid.

In a case where the polymerizable composition (I-1) or the polymerizable composition (I-2) contains a low-molecular-weight monomer as the aforementioned polymerizable compound, sometimes the monomer functions as a solvent or a dispersion medium of the specific compound (I-1), the specific compound (I-2), or the like. In this case, the polymerizable composition (I-1) or the polymerizable composition (I-2) may not contain a solvent. Even though the monomer functions as a solvent or dispersion medium of the specific compound (I-1), the specific compound (I-2), or the like, the polymerizable composition (I-1) or the polymerizable composition (I-2) may further contain a solvent for adjusting physical properties of the polymerizable composition (I-1) or (I-2).

As a solvent, an organic solvent can be used. The solvent can be used without particular limitation, as long as the solvent can satisfy the solubility of each component contained in the polymerizable composition (I-1) or the polymerizable composition (I-2) and the coating properties of the prepared polymerizable composition (I-1) or the polymerizable composition (I-2). It is preferable to select the solvent in consideration of not only the solubility or dispersibility of the specific compound (I-1) or the specific compound (I-2) and the polymerizable compound, but also the coating properties or dispersibility of colorants, such as dyes, and other ultraviolet absorbers that are incorporated into the polymerizable composition (I-1) or the polymerizable composition (I-2) as desired, the conditions and properties of a coating surface formed of the coating liquid, and ease of handling.

Examples of the organic solvent that can be incorporated into the polymerizable composition (I-1) or the polymerizable composition (I-2) as a solvent include an ester, an ether, a ketone, an aromatic hydrocarbon, and the like.

Examples of the ester include ethyl acetate, n-butyl acetate, isobutyl acetate, amyl formate, isoamyl acetate, butyl propionate, isopropyl butyrate, ethyl butyrate, butyl butyrate, methyl lactate, ethyl lactate, oxyacetic acid alkyl esters [example: methyl oxyacetate, ethyl oxyacetate, butyl oxyacetate, and the like (specifically, example thereof include methyl methoxyacetate, ethyl methoxyacetate, butyl methoxyacetate, methyl ethoxyacetate, ethyl ethoxyacetate, and the like)], 3-oxypropionic acid alkyl esters [example: methyl 3-oxypropionate, ethyl 3-oxypropionate, and the like (specifically, examples thereof include methyl 3-methoxypropionate, ethyl 3-methoxypropionate, methyl 3-ethoxypropionate, ethyl 3-ethoxypropionate, and the like)], 2-oxypropionic acid alkyl esters [example: methyl 2-oxypropionate, ethyl 2-oxypropionate, propyl 2-oxypropionate, and the like (specific examples thereof include methyl 2-methoxypropionate, ethyl 2-methoxypropionate, propyl 2-methoxypropionate, methyl 2-ethoxypropionate, ethyl 2-ethoxypropionate, and the like)], methyl 2-oxy-2-methylpropionate, ethyl 2-oxy-2-methylpropionate, and the like (specifically, examples thereof include methyl 2-methoxy-2-methylpropionate, ethyl 2-ethoxy-2-methylpropionate, and the like), methyl pyruvate, ethyl pyruvate, propyl pyruvate, methyl acetoacetate, ethyl acetoacetate, methyl 2-oxobutanoate, ethyl 2-oxobutanoate, cyclohexyl acetate, 1-methyl-2-methoxyethyl propionate, and the like.

Examples of the ether include diethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol monomethyl ether acetate (hereinafter, called PEGMEA in some cases), diethylene glycol monoethyl ether acetate (hereinafter, called ethyl carbitol acetate in some cases), diethylene glycol monobutyl ether acetate (hereinafter, called butyl carbitol acetate in some cases), propylene glycol monoethyl ether acetate, propylene glycol monopropyl ether acetate, and the like.

Examples of the ketone include methyl ethyl ketone, cyclohexanone, 2-heptanone, 3-heptanone, and the like.

Suitable examples of the aromatic hydrocarbon include toluene, xylene, and the like.

In a case where the polymerizable composition (I-1) or the polymerizable composition (I-2) contains a solvent, the polymerizable composition (I-1) or the polymerizable composition (I-2) may contain only one kind of solvent, or contain two or more kinds of solvents from the viewpoint of solubility of each component, improvement of condition of a coating surface, and the like.

In a case where the polymerizable composition (I-1) or the polymerizable composition (I-2) contains two or more kinds of solvents, it is preferable that the polymerizable composition (I-1) or (I-2) contain two or more kinds of solvents selected from the group consisting of methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, ethyl carbitol acetate, butyl carbitol acetate, propylene glycol methyl ether, and propylene glycol methyl ether acetate.

In a case where the polymerizable composition (I-1) or the polymerizable composition (I-2) contains an organic solvent as a solvent, the content of the organic solvent in the polymerizable composition (I-1) or the polymerizable composition (I-2) with respect to the total solid content in the composition is preferably within a range of 10% by mass to 80% by mass, and more preferably within a range of 15% by mass to 60% by mass.

(Surfactant)

The polymerizable composition (I-1) or the polymerizable composition (I-2) can contain a surfactant. In a case where the polymerizable composition (I-1) or the polymerizable composition (I-2) contains a surfactant, sometimes the condition and properties of a coating surface formed of the polymerizable composition (I-1) or the polymerizable composition (I-2) and the adhesiveness of the polymerizable composition (I-1) or (I-2) to a substrate can be further improved.

Examples of the surfactant include polyoxyethylene alkyl ethers such as polyoxyethylene lauryl ether, polyoxyethylene stearyl ether, polyoxyethylene cetyl ether, and polyoxyethylene oleyl ether; polyoxyethylene alkylallyl ethers such as polyoxyethylene octylphenol ether and polyoxyethylene nonylphenol ether; polyoxyethylene/polyoxypropylene block copolymers; sorbitan fatty acid esters such as sorbitan monolaurate, sorbitan monopalmitate, sorbitan monostearate, sorbitan monooleate, sorbitan trioleate, and sorbitan tristearate; nonionic surfactants such as polyoxyethylene sorbitan fatty acid esters including polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate, fluorine-based surfactants such as trade name: EFTOP EF301, EF303, and EF352 (manufactured by Mitsubishi Materials Electronic Chemicals Co., Ltd. (formerly manufactured by Gemco Co., Ltd.), trade name: MEGAFACE (registered trademark, the same shall be applied hereinafter) F171, F173, R-08, R-30, F-553, and F-554 (manufactured by DIC Corporation), trade name: FLUORAD FC430 and FC431 (manufactured by Sumitomo 3M Limited.), trade name: AsahiGuard AG710, and SURFLON (registered trademark, the same shall be applied hereinafter) S-382, SC101, SC102, SC103, SC104, SC105, and SC106 (manufactured by AGC Inc.), trade name: organosiloxane polymer KP341 (manufactured by Shin-Etsu Chemical Co., Ltd.), trade name: BYK-302, BYK-307, BYK-322, BYK-323, BYK-330, BYK-333, BYK-370, BYK-375, and BYK-378 (manufactured by BYK Chemie Japan K.K.), and the like.

In a case where the polymerizable composition (I-1) or the polymerizable composition (I-2) contains a surfactant, the polymerizable composition (I-1) or the polymerizable composition (I-2) may contain only one kind of surfactant or two or more kinds of surfactants.

In a case where the polymerizable composition (I-1) or the polymerizable composition (I-2) contains a surfactant, the content of the surfactant in the polymerizable composition (I-1) or the polymerizable composition (I-2) with respect to the content of 1 part by mass of the specific compound (I-1) or the specific compound (I-2) is preferably 0.0001 parts by mass to 5 parts by mass, more preferably 0.001 parts by mass to 2 parts by mass, and even more preferably 0.01 parts by mass to 1 part by mass.

(Colorant)

The polymerizable composition may contain a colorant. Examples of the colorant include a pigment and a dye.

Preferable examples of the pigment include inorganic pigments, such as titanium oxide, zinc oxide, carbon black, aluminum powder, ferric oxide (red iron oxide), lead chromate, molybdate orange, chrome yellow, yellow iron oxide, ochre, ultramarine, and cobalt green, and organic pigments, such as azo-based, naphthol-based, pyrazolone-based, anthraquinone-based, perylene-based, quinacridone-based, disazo-based, isoindolinone-based, benzimidazole-based, phthalocyanine-based, and quinophthalone-based pigments.

Preferable examples of the dye include organic dyes, such as anthraquinone-based, quinophthalone-based, methine-based, phthalocyanine-based, and perylene-based dyes.

In a case where the polymerizable composition of the present disclosure contains a colorant, the polymerizable composition may contain only one kind of colorant or two or more kinds of colorants.

The content of the colorant is appropriately adjusted depending on the purpose. In a case where the polymerizable composition contains a colorant, the content of the colorant with respect to the total solid content of the polymerizable composition is preferably 0.1% by mass to 10% by mass, and more preferably 0.1% by mass to 1% by mass.

[Ultraviolet Cut Film (First Aspect)]

A first aspect of the ultraviolet cut film of the present disclosure is a cured substance of the polymerizable composition (I-1) or the polymerizable composition (I-2) of the present disclosure described above.

The ultraviolet cut film of the present disclosure can be obtained by curing the polymerizable composition (I-1) or the polymerizable composition (I-2) of the present disclosure described above. The cured substance of the polymerizable composition (I-1) or the polymerizable composition (I-2) can be obtained by forming a polymerizable composition layer by using the polymerizable composition (I-1) or the polymerizable composition (I-2) and curing the polymerizable composition layer by the application of energy. The polymerizable composition layer may be formed on a desired support.

Furthermore, a desired molding die may be filled with the polymerizable composition (I-1) or the polymerizable composition (I-2), and the composition may be cured. Alternatively, a semi-cured substance of the polymerizable composition (I-1) or the polymerizable composition (I-2) may be disposed in a desired mold and cured.

Examples of the application of energy include methods such as light irradiation and heating. Among these, light irradiation is preferable, and ultraviolet irradiation is more preferable. In a case where light irradiation is adopted as the application of energy, it is preferable that the polymerizable composition (I-1) or the polymerizable composition (I-2) contain a photoradical polymerization initiator.

In a case where the polymerizable composition (I-1) or the polymerizable composition (I-2) is disposed in a molding die and cured, as a preferable aspect, heating is performed as the application of energy.

In a case where heating is adopted as the application of energy, it is preferable that the polymerizable composition (I-1) or the polymerizable composition (I-2) contain a thermal radical polymerization initiator.

In a case where the polymerizable composition (I-1) or the polymerizable composition (I-2) contains a solvent, from the viewpoint of further improving curing properties, it is preferable to reduce in advance the amount of solvent contained in the polymerizable composition layer before the application of energy. As a method for reducing the amount of solvent, a method of drying the polymerizable composition layer is preferable.

In a case where drying is to be performed, examples of the drying method include known methods, such as a method of blasting the polymerizable composition layer with hot air, a method of passing the polymerizable composition layer through a drying zone at a predetermined controlled temperature, a method of drying the polymerizable composition layer by using a heater comprising a transport roll, and the like.

For the purpose of accelerating the curing reaction in the polymerizable composition layer, it is possible to use a method of raising the temperature of the polymerizable composition layer during curing. From the viewpoint of accelerating the curing reaction, the temperature of the polymerizable composition layer is preferably 25° C. to 100° C., more preferably 30° C. to 80° C., and even more preferably 40° C. to 70° C.

In a case where the ultraviolet cut film is formed by putting the polymerizable composition (I-1) or the polymerizable composition (I-2) in a molding die and curing the composition, the ultraviolet cut film can be obtained by directly filling a molding die with the polymerizable composition (I-1) or the polymerizable composition (I-2) and then curing the composition (I-1) or (I-2) by the application of energy.

Furthermore, the ultraviolet cut film may be formed by the application of energy under conditions where a semi-cured substance is obtained before the polymerizable composition layer is fully cured, putting the obtained semi-cured substance in a molding die, and then fully curing the semi-cured substance.

By directly installing the polymerizable composition (I-1) or the polymerizable composition (I-2) of the present disclosure or installing a semi-cured substance of the polymerizable composition (I-1) or the polymerizable composition (I-2) that is obtained by performing light irradiation, heating, or the like on the polymerizable composition (I-1) or the polymerizable composition (I-2) in a molding die, and then applying energy thereto, it is possible to form an ultraviolet cut film in any shape in accordance with the molding die.

In a case where light irradiation is performed as the application of energy, for example, an ultraviolet lamp can be used. The light irradiation dose is preferably in a range of 10 mJ/cm² to 1,000 mJ/cm². In a case where ultraviolet rays are radiated at this irradiation dose, the polymerizable composition layer is suitably cured, which makes it possible to efficiently obtain an ultraviolet cut film as a cured substance.

In performing ultraviolet irradiation, for the purpose of suppressing curing inhibition caused by oxygen and further facilitating surface curing of the polymerizable composition layer, the region irradiated with ultraviolet rays can be purged with an inert gas such as a nitrogen gas so that the oxygen concentration is reduced. In a case where the oxygen concentration in the region irradiated with ultraviolet rays is to be reduced, it is preferable that the oxygen concentration in the region irradiated with ultraviolet rays be reduced to 1% by mass or less.

In a case where heating is performed as the application of energy for inducing thermal polymerization, the heating time is preferably 30 seconds to 1,000 seconds, more preferably 30 seconds to 500 seconds, and even more preferably 60 seconds to 300 seconds.

The heating temperature is appropriately selected depending on the makeup of the polymerizable composition (I-1) or the polymerizable composition (I-2), the size and shape of the target cured substance, and the like. The heating temperature is preferably 60° C. or higher, which is 70° C. to 200° C., for example. For instance, the heating temperature is preferably 80° C. to 180° C.

In a case where the polymerizable composition (I-1) or the polymerizable composition (I-2) is to be thermally polymerized by heating, the thermal polymerization may be performed in an atmosphere such as air or an atmosphere purged with an inert gas such as a nitrogen gas. From the viewpoint of curing properties, an atmosphere purged with an inert gas is preferable, and an atmosphere purged with nitrogen until the oxygen concentration reaches 1% by mass or less is more preferable.

The thickness of the ultraviolet cut film is not particularly limited, and can be arbitrarily selected within a range where preferable content of the specific compound (I-1) or the specific compound (I-2), preferable ultraviolet cut properties, preferable visible light transmittance, and the like can be achieved.

The thickness of the ultraviolet cut film can be, for example, in a range of 5 μm to 2,500 and is preferably in a range of 20 μm to 500 In a case where the thickness of the ultraviolet cut film is within this range, it is easy to obtain desired ultraviolet cut properties and visible light transmittance and to handle the ultraviolet cut film.

The first aspect of the ultraviolet cut film is a cured substance of the polymerizable composition (I-1) or the polymerizable composition (I-2) of the present disclosure described above. Therefore, due to the makeup of the polymerizable composition (I-1) or the polymerizable composition (I-2), the content of the specific compound (I-1) or the specific compound (I-2) in the ultraviolet cut film is preferably within a range of 0.005 mmol (millimoles)/m² to 0.1 mmol/m², and more preferably in a range of 0.01 mmol/m² to 0.05 mmol/m².

In a case where the polymerizable composition (I-1) or the polymerizable composition (I-2) is formed into a cured substance by being cured using a molding die, an ultraviolet cut film having any shape can be formed. Therefore, the thickness is not always uniform, but the average thickness of the cured substance can be in the preferable range described above.

That is, because the ultraviolet cut film can be manufactured in any shape, the ultraviolet cut film of the present disclosure can be used in various fields that require cutting of ultraviolet rays. The ultraviolet cut film of the present disclosure can be manufactured in any shape, which makes it possible to easily obtain a dome-shaped ultraviolet cut film, an ultraviolet cut film in the form of a cover of a lighting device such as a headlight, a spectacle lens, a contact lens, and the like.

The ultraviolet cut film of the first aspect of the present disclosure contains the specific compound (I-1) or the specific compound (I-2). Therefore, the ultraviolet cut film excellently cuts off the wavelength region including at least the long wavelength region of ultraviolet light and the short wavelength region of visible light. As described above, in a case where the polymerizable composition (I-1) or the polymerizable composition (I-2) further contains other ultraviolet absorbers, the ultraviolet cut film of the first aspect of the present disclosure can be an ultraviolet cut film that can effectively cut off the desired wavelength region of ultraviolet rays.

[Laminate (First Aspect)]

A first aspect of the laminate of the present disclosure has a support and the aforementioned ultraviolet cut film (first aspect).

By forming the ultraviolet cut film which is a cured substance of the polymerizable composition (I-1) or the polymerizable composition (I-2) of the present disclosure on any support so as to form a laminate having the support and the ultraviolet cut film, it is possible to obtain a laminate having an ultraviolet cut film on any support.

The support is not particularly limited. However, it is preferable that the laminate of the present disclosure have a transparent support, because then the characteristics of the ultraviolet cut film are easily exhibited.

(Transparent Support)

Suitable examples of the transparent support of the laminate of the present disclosure include a glass plate and a general resin film.

Examples of the resin constituting the resin film that can be used as the transparent support include polyester such as polyethylene terephthalate (PET), polyethylene naphthalate (PEN), polybutylene terephthalate (PBT), and polycyclohexanedimethylene terephthalate (PCT), polypropylene (PP), polyethylene (PE), polyvinyl chloride (PVA), cellulose triacetate (TAC), polymethylmethacrylate (PMMA), polystyrene (PS), polycarbonate (PC), and the like. Among these, in view of general-purpose properties, PET is preferable.

“Transparent” means that the average transmittance in the visible light region, for example, the wavelength range of 450 nm to 750 nm is 80% or more.

The visible light transmittance is determined by measuring a spectroscopic spectrum with the aforementioned ultraviolet/visible spectrophotometer UV-1800 (trade name) manufactured by Shimadzu Corporation, and calculating the amount of transmitted visible light in the above wavelength region to the amount of incidence visible light in the same wavelength region.

In a case where the laminate of the first aspect of the present disclosure has a transparent support, the laminate cuts off only ultraviolet rays without hindering the transmittance visible light.

Furthermore, in a case where the laminate has a transparent support, the laminate can have higher strength than a laminate using only an ultraviolet cut film.

The thickness of the transparent support is not particularly limited, and can be appropriately selected depending on the purpose of use of the laminate.

The thickness of the transparent support can be, for example, in a range of 100 μm to 10 mm in general.

The shape of the support can also be arbitrarily selected. For example, the laminate can have the ultraviolet cut film of the present disclosure on a lens-shaped transparent support.

The laminate may have a two-layer structure consisting of a support and an ultraviolet cut film, or may have a structure consisting of three or more layers including other layers.

In a case where the laminate has a structure consisting of three or more layers, examples of layers other than the ultraviolet cut film include an adhesive layer, a surface protective layer, (such as an overcoat layer or a hardcoat layer), a reflective layer (such as a dielectric multi-layer film or a photonic crystal film), a colored layer, and the like.

The support is not limited to the transparent support, and any support can be used.

For example, in a case where a resin molded article or the like is used as a support, and an ultraviolet cut film is disposed on the support to form a laminate, it is possible to reduce the influence of ultraviolet rays on the resin molded article and to further improve the durability of the resin molded article.

The polymerizable composition (I-1) or polymerizable composition (I-2), ultraviolet cut film, and laminate of the present disclosure described above are suitably used for cutting off ultraviolet rays. The use will be described later.

[Compound Represented by General Formula (II)]

The compound represented by General Formula (II) of the present disclosure (hereinafter, called specific compound (II) in some cases) is a novel compound.

The specific compound (II) has excellent ultraviolet cut properties.

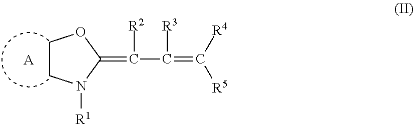

In General Formula (II), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, and the 5- or 6-membered ring may be further condensed.

Here, at least one of R′, R², R³, R⁴, R⁵, or A contains a substituent selected from the group consisting of General Formula (III) and General Formula (IV), and in a case where at least one of R′, R², R³, R⁴, R⁵, or A contains a substituent selected from the group consisting of General Formula (III) and General Formula (IV), at least one of R′, R², R³, R⁴, R⁵, or A may be a substituent selected from the group consisting of General Formula (III) and General Formula (IV).

R¹, R², R³, and A in General Formula (II) have the same definition as R¹, R², R³, and A in General Formula (I-1) and General Formula (I-2), and preferable examples thereof are also the same.

A in General Formula (II) is preferably a benzene ring or a naphthalene ring.

In General Formula (II), R⁴ and R⁵ each independently represent an electron-withdrawing group.

Specifically, as the electron-withdrawing group represented by R⁴ and R⁵, a cyano group, an alkylsulfonyl group, an arylsulfonyl group, an alkoxycarbonyl group, a carbamoyl group, an alkylcarbonyl group, an arylcarbonyl group, and the like are preferable from the view point of excellent absorption characteristics of the compound, and a cyano group, an alkylsulfonyl group, an arylsulfonyl group, an alkoxycarbonyl group, and the like are more preferable.

Examples of the electron-withdrawing group represented by R⁴ and R⁵ in General Formula (II) are the same as the examples of R⁴ and R⁵ in General Formula (I-1) and General Formula (I-2).

Examples of the compound represented by General Formula (II) include a compound represented by General Formula (II-1) or General Formula (II-2) containing a preferable electron-withdrawing group.

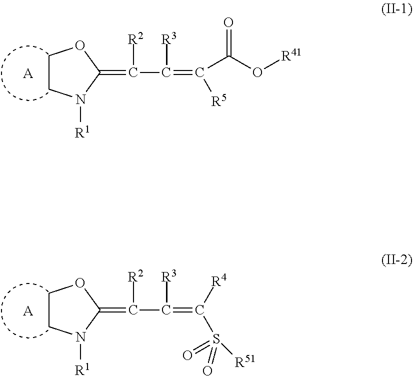

R¹, R², R³, and A in General Formula (II-1) and General Formula (II-2) have the same definition as R¹, R², R³, and A in General Formula (II), and preferable examples thereof are also the same. R⁴¹ and R⁵¹ each independently represent a monovalent electron-withdrawing group, and at least one of R¹, R², R³, R⁴, R⁵, R⁴¹, R⁵¹, or A contains a substituent selected from the group consisting of General Formula (III) and General Formula (IV). In a case where at least one of R¹, R², R³, R⁴, or R⁵ contains a substituent selected from the group consisting of General Formula (III) and General Formula (IV), at least one of R¹, R², R³, R⁴, or R⁵ may be a substituent selected from the group consisting of General Formula (III) and General Formula (IV).

R⁴ and R⁵ in General Formula (II) may form a ring by being bonded to each other, or may not form a ring. From the viewpoint of further improving absorptivity for a short wavelength region of visible light and obtaining a sharper absorption peak, it is preferable that R⁴ and R⁵ be not bonded to each other to form a ring.

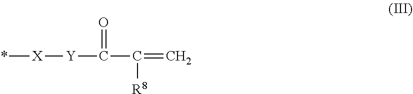

In General Formula (III), X represents a single bond or an alkylene group, Y represents a single bond, —O—, or —NR¹⁴—, and R¹⁴ represents a hydrogen atom or an alkyl group. R⁸ represents a hydrogen atom or an alkyl group. * Represents a binding position.

In General Formula (III), R⁸ represents a hydrogen atom or an alkyl group.

In a case where R⁸ represents an alkyl group, as the alkyl group, methyl and methoxymethyl are preferable. Particularly, from the viewpoint of further improving the polymerization properties of the compound, R⁸ is preferably a hydrogen atom or a methyl group.

In General Formula (III), X represents a single bond or an alkylene group. X is preferably an alkylene group having 1 to 20 carbon atoms in total, and more preferably an alkylene group having 1 to 8 carbon atoms.

In a case where X represents an alkylene group, the alkylene group may have a substituent such as a methyl group, an ethyl group, or a hydroxyl group, or may be interrupted by an oxygen atom or a sulfur atom.

Specifically, examples of X include methylene, ethylene, methylethylene, propylene, 2-hydroxypropylene, tetramethylene, hexamethylene, octamethylene, —(OCH₂CH₂)ₙ— (n represents an integer of 1 to 4), —(OCH₂CHCH₃)ₙ— (n represents an integer of 1 to 4), and the like.

In General Formula (III), Y represents a single bond, —O—, or NR¹⁴—. The alkyl group represented by R¹⁴ is preferably an alkyl group having 1 to 8 carbon atoms. Specifically, examples thereof include methyl, ethyl, propyl, butyl, hexyl, and octyl.

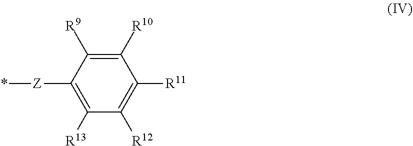

In General Formula (IV), R⁹, R¹⁰, R¹¹, R¹² and R¹³ each independently represent a hydrogen atom, a halogen atom, an alkyl group, or an alkoxy group, and Z represents a single bond or an alkylene group. * Represents a binding position. Here, at least one of R⁹, R¹⁰, R¹¹, R¹², or R¹³ represents a vinyl group.

In General Formula (IV), R⁹, R¹⁰, R¹¹, R¹² and R¹³ represent a hydrogen atom, a halogen atom, an alkyl group, or an alkoxy group.

In a case where R⁹, R¹⁰, R¹¹, R¹² and R¹³ represent a halogen atom, the halogen atom is preferably a fluorine atom, a chlorine atom, a bromine atom, and an iodine atom, and more preferably a fluorine atom and a chlorine atom.

In a case where R⁹, R¹⁰, R¹¹, R¹² and R¹³ represent an alkyl group, as the alkyl group, methyl, ethyl, and the like are preferable.

In a case where R⁹, R¹⁰, R¹¹, R¹² and R¹³ represent an alkoxy group, examples of the alkoxy group include methoxy, ethoxy, methylenedioxy, and the like.

Especially, it is preferable that one of R⁹, R¹⁰, R¹¹, R¹², and R¹³ be a vinyl group and the other four be hydrogen atoms.

In General Formula (IV), Z represents a single bond or an alkylene group.

In a case where Z represents an alkylene group, as the alkylene group, an alkylene group having 1 to 20 carbon atoms in total is preferable, and an alkylene group having 1 to 8 carbon atoms in total is particularly preferable. These alkylene groups may have a substituent such as a methyl group, an ethyl group, or a hydroxyl group, or may have a hetero atom selected from an oxygen atom and a sulfur atom in the carbon chain of the alkylene group. Specifically, examples of thereof include methylene, ethylene, methylethylene, propylene, 2-hydroxypropylene, tetramethylene, hexamethylene, octamethylene, —(OCH₂CH₂)ₙ— (n represents an integer of 1 to 4), —(OCH₂CHCH₃)ₙ— (n represents an integer of 1 to 4), and the like.

It is preferable that the specific compound (II) have absorption maximum in a wavelength range of 390 nm to 430 nm in ethyl acetate.

The absorption maximum of the specific compound (II) in ethyl acetate is measured in the same manner as the aforementioned method for measuring the absorption maximum of the specific compound (I-1) or the specific compound (I-2) in ethyl acetate.

The specific compound (II) contains a polymerizable group in the molecule. Therefore, the specific compound (II) is effectively immobilized in a cured substance of the polymerizable composition containing the specific compound (II) [hereinafter, called polymerizable composition (II) in some cases] or in a cured substance of a resin composition containing the specific compound (II) that will be described later. As a result, elution and the like of the specific compound (II) from the cured substance are inhibited. Accordingly, both the polymerizable composition (II) and resin composition containing the specific compound (II) can form a cured substance in which bleed out, outflow, precipitation, and the like of the specific compound (II) are suppressed.

Specific examples of the specific compound (II) [exemplary compound 1 to exemplary compound 62] will be shown below. The present disclosure is not limited to the following specific examples.

In the following exemplary compounds, Me represents a methyl group, and Et represents an ethyl group.

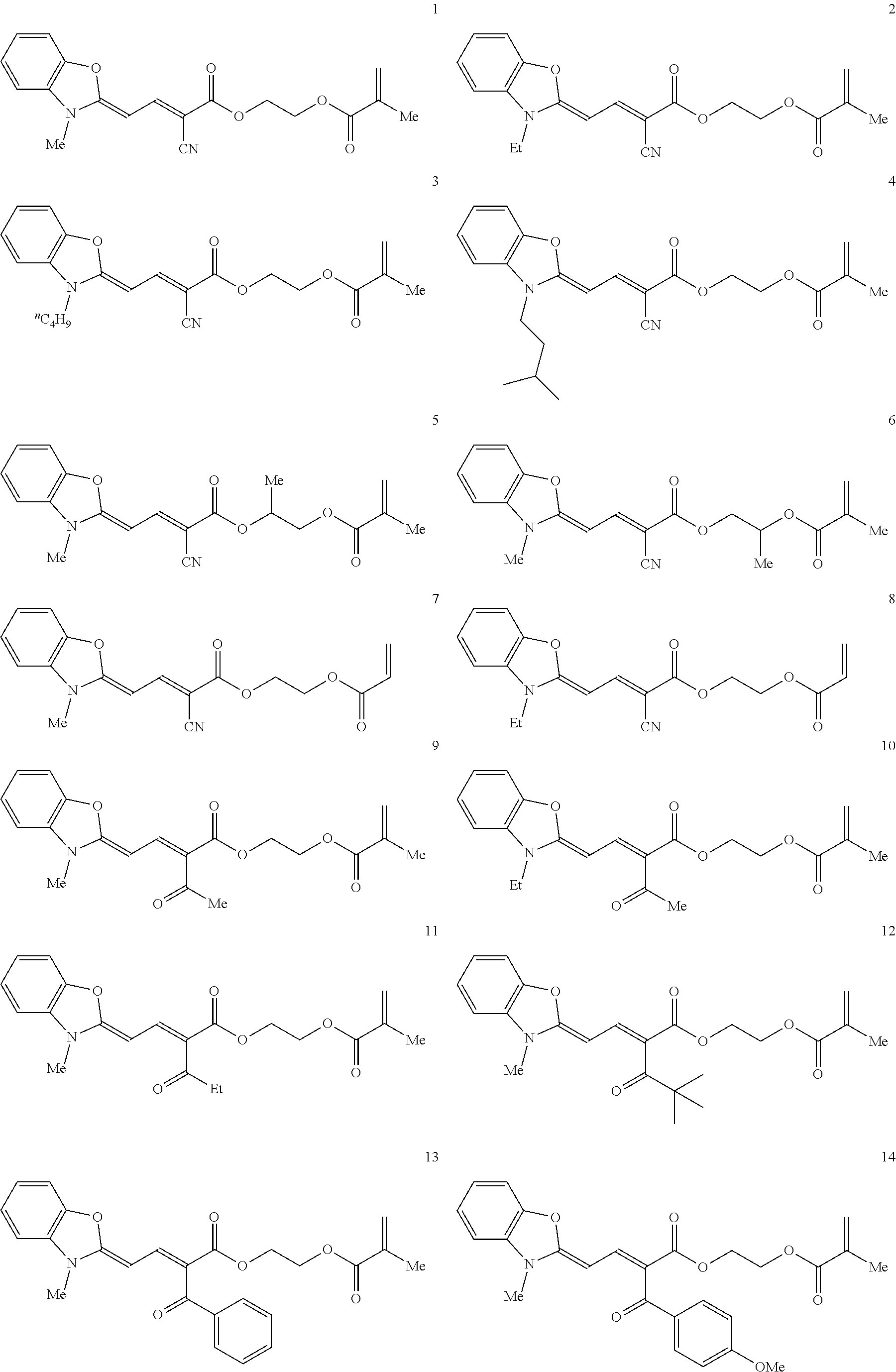

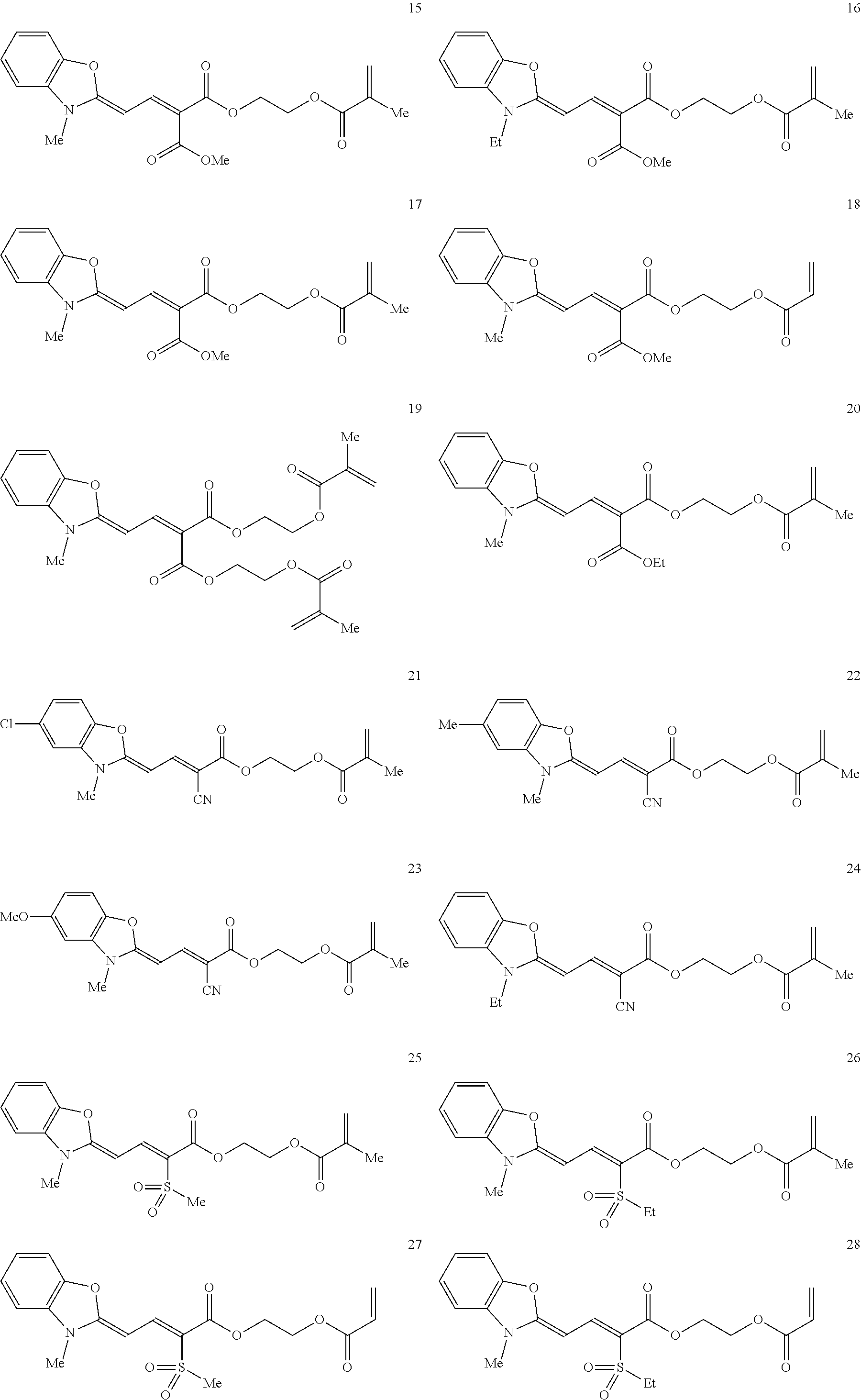

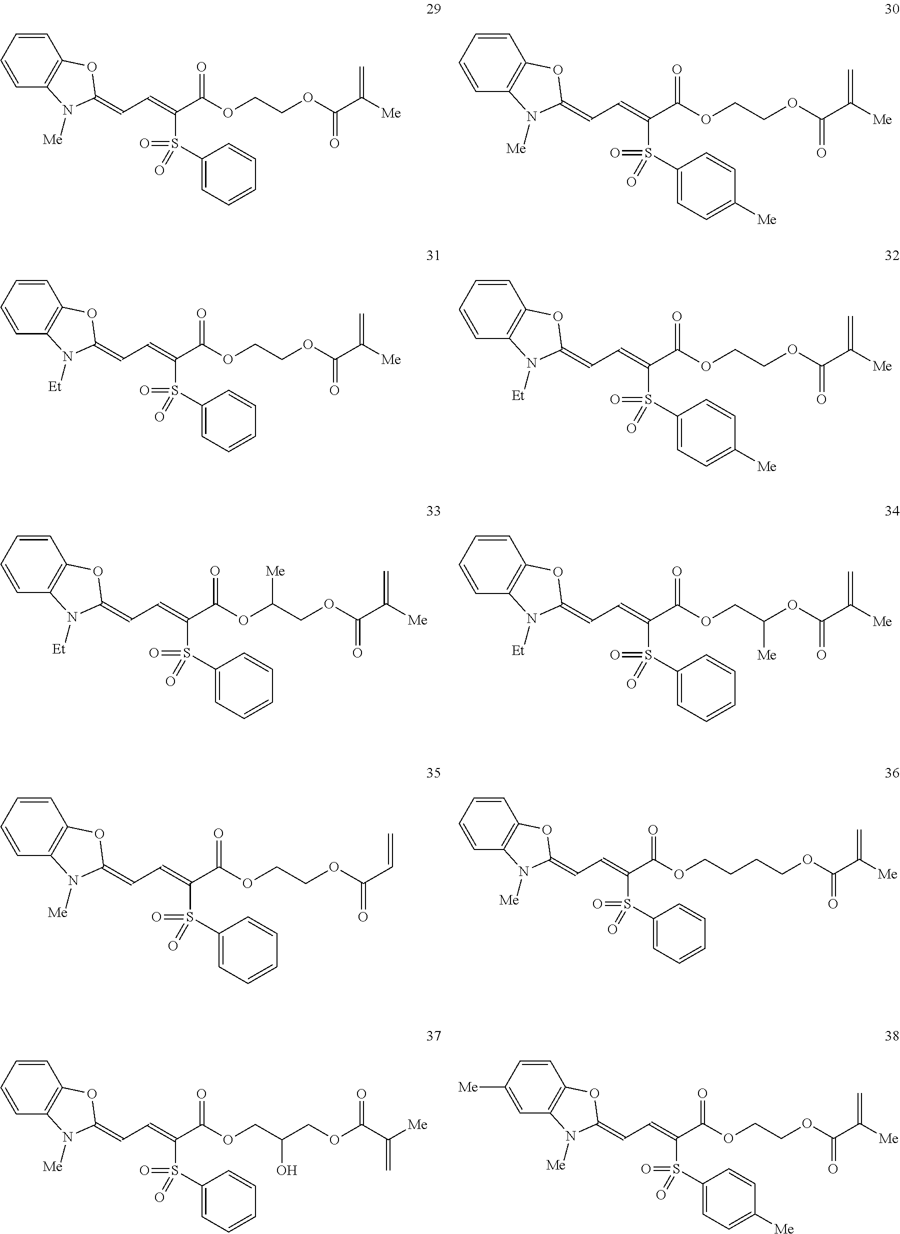

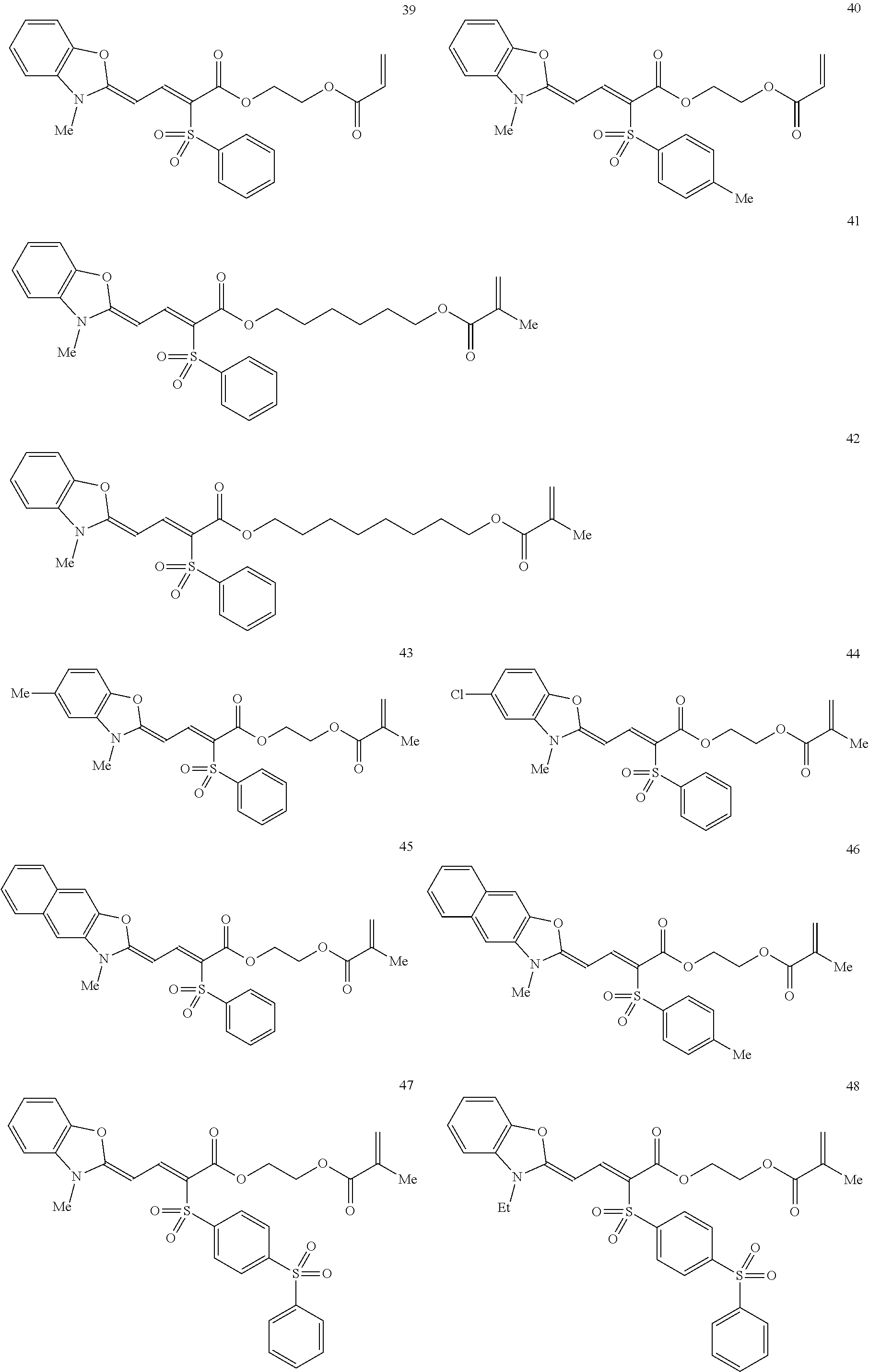

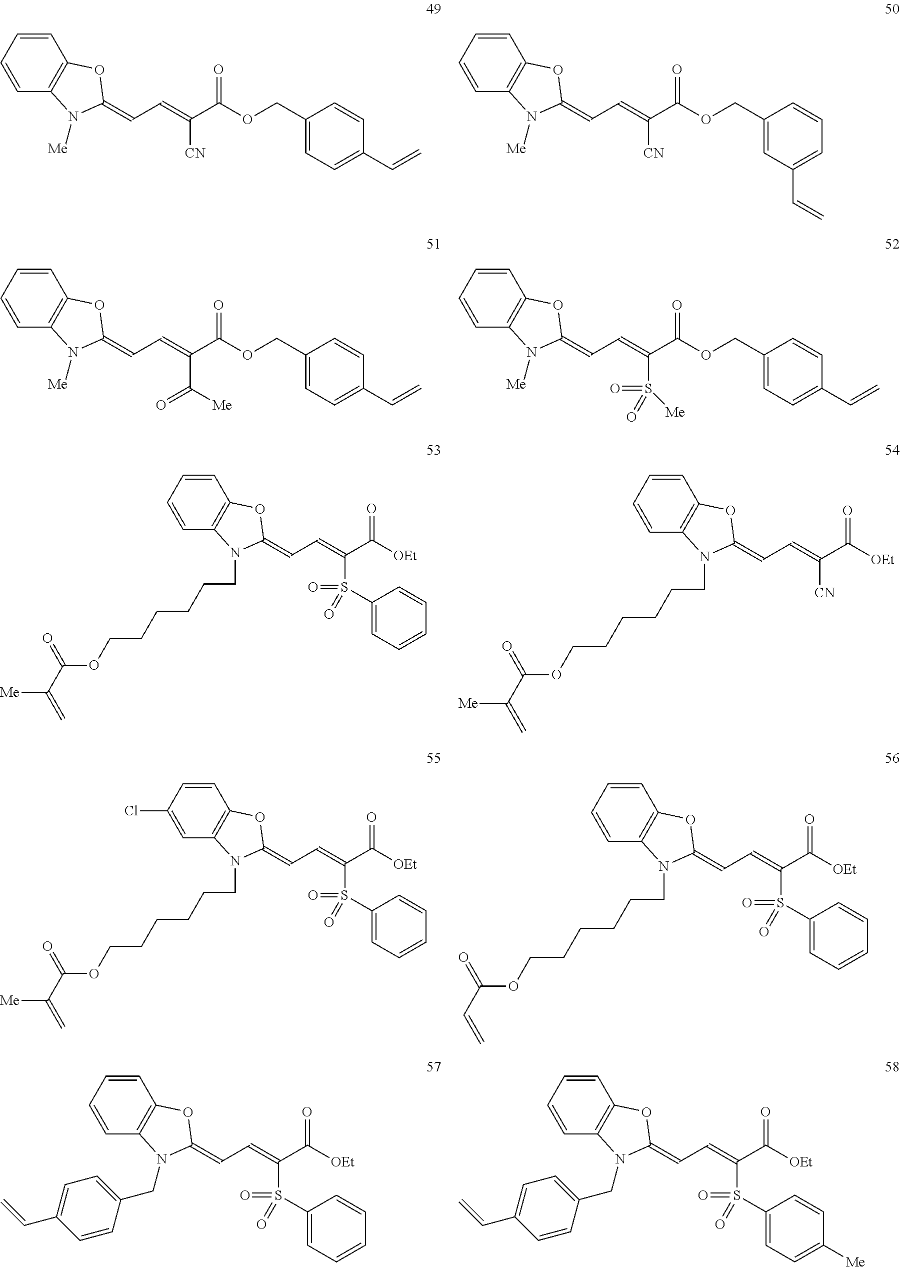

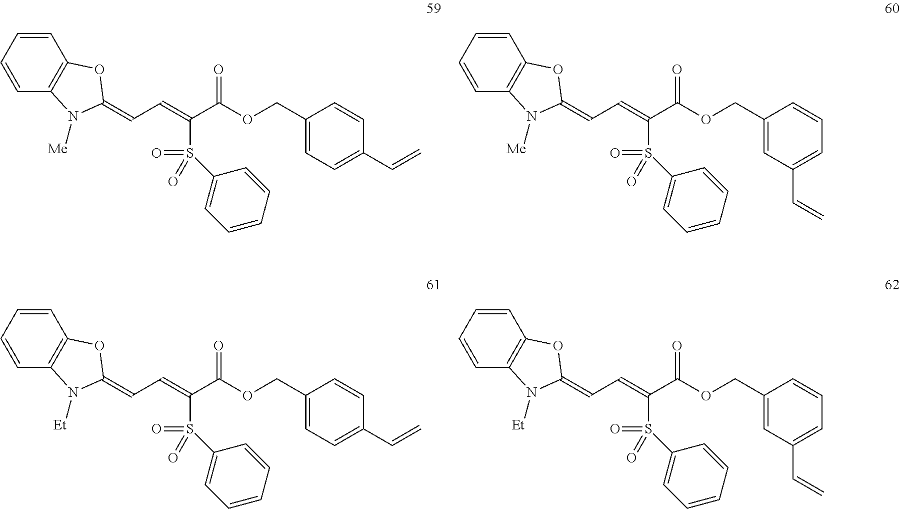 [SMILES: C=Cc1ccc(COC(=O)/C(=C/C=C2\Oc3ccccc3N2C)S(=O)(=O)c2ccccc2)cc1 ; C=Cc1cccc(COC(=O)/C(=C/C=C2\Oc3ccccc3N2C)S(=O)(=O)c2ccccc2)c1 ; C=Cc1ccc(COC(=O)/C(=C/C=C2\Oc3ccccc3N2CC)S(=O)(=O)c2ccccc2)cc1 ; C=Cc1cccc(COC(=O)/C(=C/C=C2\Oc3ccccc3N2CC)S(=O)(=O)c2ccccc2)c1]

In a case where the specific compound (II) in the present disclosure, for example, the above exemplary compounds and the like include at least a geometric isomer or a tautomer, the specific compound (II) of the present disclosure includes both the geometric isomer and tautomer.

Details of the method for synthesizing the specific compound (II) will be described later in Examples.

[Polymer]

The polymer of the present disclosure contains a constitutional unit derived from the aforementioned specific compound (II).

Containing the constitutional unit derived from the specific compound (II), the polymer of the present disclosure (hereinafter, called a specific polymer in some cases) has excellent ultraviolet cut properties.

It is preferable that the specific polymer have absorption maximum in a wavelength range of 390 nm to 430 nm in ethyl acetate.

The specific polymer may be a homopolymer containing only the constitutional unit derived from the specific compound (II), or a copolymer containing the constitutional unit derived from the specific compound (II) and other monomers. From the viewpoint of higher ultraviolet cut properties, the specific polymer is preferably a homopolymer consisting of only the constitutional unit derived from the specific compound (II). Furthermore, from the viewpoint of making it possible to adjust the physical properties of the polymer, the specific polymer is preferably a copolymer containing the constitutional unit derived from the specific compound (II) and a constitutional unit derived from other monomers.

(Other Monomer)

Examples of other monomers that the specific polymer can contain include the polymerizable compounds that the polymerizable composition (I-1) or the polymerizable composition (I-2) can contain. Examples of the polymerizable compounds include a monomer and the like.

Particularly preferable examples of those other monomers include a (meth)acrylate monomer, a styrene monomer, and the like.

From the viewpoint of higher ultraviolet cut properties, a copolymerization ratio of constitutional unit derived from the specific compound (II)/other monomers in the specific polymer is preferably 1/1 to 1/500 (mass ratio), and more preferably 1/10 to 1/500 (mass ratio).

Examples of other monomers that the polymer can contain include a constitutional unit derived from other ultraviolet absorbers which are different from the specific compound (II) and have a double bond. In a case where the polymer contains, as a constitutional unit, other ultraviolet absorbers having a double bond in the molecule, the polymer can cut off not only ultraviolet rays due to the specific compound (II) but also ultraviolet rays having different wavelengths due to those other ultraviolet absorbers. Furthermore, because those other ultraviolet absorbers have a double bond, not only the bleed out and the like of the specific compound (II) but also the bleed out and the like of those other ultraviolet absorbers from the polymer can be effectively suppressed.

The type and content of those other ultraviolet absorbers are appropriately selected in consideration of the ability to cut off ultraviolet rays of target wavelengths. The content of those other ultraviolet absorbers in the polymer can be 0.2% by mass to 10% by mass with respect to the total amount of the polymer.

The weight-average molecular weight of the polymer of the present disclosure can be appropriately selected depending on the purpose of the resin composition containing the polymer of the present disclosure that will be described later.

Specifically, the weight-average molecular weight of the polymer can be, for example, 8,000 to 100,000, and is preferably in a range of 10,000 to 50,000.

The weight-average molecular weight of the polymer and the polymer compound contained in the resin composition that will be described later can be measured by the following method based on gel permeation chromatography (GPC) or the like using an aqueous eluent (such as tetrahydrofuran).

The weight-average molecular weight is measured by gel permeation chromatography (GPC) as a polystyrene-equivalent molecular weight under the following conditions. The calibration curve is prepared from 6 samples of “Standard sample TSK standard, polystyrene” manufactured by Tosoh Corporation: “F-128”, “F-40”, “F-20”, “F-4”, “F-1”, and “A-2500”.

<Conditions>
GPC: HLC (registered trademark)-8220 (manufactured by Tosoh Corporation)
Column: HZM-N
Eluent: selectable from tetrahydrofuran (THF) and N-methyl-2-pyrrolidone (NMP), THF will be used for dissolution.
Sample concentration: 0.5 mass/volume %
Flow rate: 0.35 ml/min
Sample injection amount: 10 μl
Measurement temperature: 40° C.
Using differential refractometer (RI) detector

[Polymerizable Composition (II)]

A third aspect of the polymerizable composition of the present disclosure contains the aforementioned specific compound (II). Containing a polymerizable group in the molecule, the specific compound (II) also functions as a polymerizable compound. The third aspect of the polymerizable composition containing the specific compound (II) will be called “polymerizable composition (II)” as described above.

If necessary, the polymerizable composition (II) may contain other polymerizable compounds different from the specific compound (II). Examples of those other polymerizable compounds include the polymerizable compounds described above regarding the polymerizable compound (I-1) and the polymerizable composition (I-2), and preferable examples thereof are also the same.

The content of the specific compound (II) in the polymerizable composition (II) is not particularly limited, and may be appropriately selected depending on the purpose.

From the viewpoint of excellent balance between the ultraviolet cut effect and the visibility through a cured substance in a case where the polymerizable composition is made into a cured substance, the content of the specific compound (II) in the polymerizable composition (II) is preferably in a range of 0.005 mmol (millimoles)/m² to 0.1 mmol/m², and more preferably in a range of 0.01 mmol/m² to 0.05 mmol/m².

In addition, from the viewpoint of excellent balance between the ultraviolet cut effect and the visibility through a cured substance in a case where the polymerizable composition (II) is made into a cured substance, the content of the specific compound (II) with respect to the total solid content of the polymerizable composition (II) is preferably 0.01% by mass to 5% by mass, more preferably 0.1% by mass to 3% by mass, and even more preferably 0.3% by mass to 1% by mass.

The polymerizable composition (II) can contain optional components other than the polymerizable compound (called other components in some cases), in addition to the specific compound (II) and other polymerizable compounds as optional components.

Those other components that the polymerizable composition (II) can contain are the same as optional components (other components) described above regarding the polymerizable composition (I-1) or the polymerizable composition (I-2), and preferable examples thereof are also the same.

It is particularly preferable that the polymerizable composition (II) additionally contain, as other components, ultraviolet absorbers other than the specific compound (II).

In a case where the polymerizable composition (II) additionally contains ultraviolet absorbers other than the specific compound (II) (other ultraviolet absorbers), the wavelength region of ultraviolet rays that the polymerizable composition (II) can cut off can be adjusted.

Examples of those other ultraviolet absorbers are the same as the examples of other ultraviolet absorbers described above regarding the polymerizable composition (I-1) and the polymerizable composition (I-2), and preferable examples thereof are also the same. Particularly, it is preferable that the polymerizable composition (II) contain benzotriazole-based ultraviolet absorbers as other ultraviolet absorbers.

Containing the specific compound (II), the polymerizable composition (II) can exhibit excellent ultraviolet cut properties just as the polymerizable composition (I-1) and the polymerizable composition (I-2). Therefore, the polymerizable composition (II) has a wide range of applications.

Having a polymerizable group in the molecule, the specific compound (II) easily interacts with and is easily bonded to a cured substance containing the specific compound (II). Consequently, the specific compound (II) as an ultraviolet cut compound is immobilized in the cured substance which will be described later, and brings about an effect of suppressing the undesirable bleed out, precipitation, and elution of ultraviolet cut components.

[Resin Composition]

The first aspect of the resin composition of the present disclosure contains the specific compound (II) and a polymer compound.

In the resin composition of the present disclosure, the polymer compound functions as a film forming compound. Furthermore, the polymer compound can also function as a substrate of a resin molded article. The polymer compound contained in the resin composition may or may not have a polymerizable group.

(Specific Compound (II))

The content of the specific compound (II) in the resin composition is not particularly limited, and may be appropriately selected depending on the purpose.

From the viewpoint of excellent balance between the ultraviolet cut effect and the visibility through a cured substance in a case where the resin composition is made into a cured substance, the content of the specific compound (II) in the resin composition is preferably in a range of 0.005 mmol (millimoles)/m² to 0.1 mmol/m², and more preferably in a range of 0.01 mmol/m² to 0.05 mmol/m².

In addition, from the viewpoint of excellent balance between the ultraviolet cut effect and the visibility through a cured substance in a case where the resin composition is made into a cured substance, the content of the specific compound (II) with respect to the total solid content of the resin composition is preferably 0.01% by mass to 5% by mass, more preferably 0.1% by mass to 3% by mass, and even more preferably 0.3% by mass to 1% by mass.

(Polymer Compound)

The polymer compound that can be incorporated into the resin composition can be appropriately selected depending on the purpose of use of the resin composition. That is, the type, molecular weight, and the like of the polymer compound may be selected in consideration of the purpose of use, workability, and required strength.

For example, in a case where the resin composition is to be used for spectacle lenses, contact lenses, and the like, a resin having excellent transparency may be selected and used.

Furthermore, in a case where the resin composition is to be used for ultraviolet cut window glass, a windshield for vehicles, and the like, a resin having excellent strength and durability is selected.

In a case where a thermoplastic resin is used as the polymer compound, the resin composition can be molded by heating. Furthermore, in a case where a thermosetting resin is used as the polymer compound, a cured substance having excellent strength and durability can be formed.

One of the examples of the polymer compound is a polymer or copolymer containing the polymerizable compound exemplified above regarding the polymerizable composition (I-1) and the polymerizable composition (I-2) as a constitutional unit, and the like.

The molecular weight of the polymer compound can be appropriately selected depending on the purpose of the resin composition.

Specifically, the weight-average molecular weight of the polymer compound can be, for example, 8,000 to 500,000, and is preferably in a range of 10,000 to 50,000.

The weight-average molecular weight of the polymer compound can be measured by the method described above.

In a case where the resin composition of the present disclosure is used for a spectacle lens, it is preferable to select the polymer compound to be used in consideration of refractive index. The polymer compound (resin) used for a spectacle lens may be a thermoplastic resin or a thermosetting resin, as long as the resin satisfies the required physical properties such as transparency, refractive index, workability, and strength after curing.

By introducing a halogen atom other than fluorine, an aromatic ring, a sulfur atom, and the like into the resin, it is possible to further increase the refractive index of the resin.

Examples of the thermoplastic resin that can be used for a spectacle lens include one or more kinds of resins selected from polycarbonate; an acrylic resin such as polymethylmethacrylate (PMMA); and the like.

Commercially available products may be used as the thermoplastic resin used for a spectacle lens. Examples of the commercially available products include a polycarbonate resin composition (CALIBRE 200-13: trade name, Sumitomo Dow Limited), a diethylene glycol bisallyl carbonate resin (CR-39: trade name, manufactured by PPG Industries, Inc.), and the like.

From the viewpoint of molding properties and handleability, the content of the polymer compound in the resin composition of the present disclosure with respect to the total solid content of the resin composition is preferably 50% by mass to 99% by mass, and more preferably 70% by mass to 99% by mass.

Examples of a second aspect of the resin composition of the present disclosure include a resin composition containing the polymer of the present disclosure described above.

The polymer of the present disclosure described above is a polymer containing a constitutional unit derived from the specific compound (II), that is, a polymer compound containing a constitutional unit derived from the specific compound (II). Therefore, the polymer compound itself has excellent ultraviolet cut properties, the polymer of the present disclosure can be directly used as a molding material, and the obtained molded article has excellent ultraviolet cut properties.

In the resin composition, from the viewpoint of ultraviolet cut properties and handleability, the content of the polymer of the present disclosure with respect to the total solid content of the resin composition is preferably 0.1% by mass to 99% by mass, and more preferably 10% by mass to 99% by mass.

All of the resin compositions of the present disclosure described above have the specific compound (II) or a constitutional unit derived from the specific compound (II). Therefore, the resin compositions have excellent ultraviolet cut properties.

The resin composition of the present disclosure can contain other components different from the specific compound (II) and the polymer compound or the polymer containing a constitutional unit derived from the specific compound (II), as long as the effects of the resin composition are not impaired.

As those other components, any components generally used in a resin composition can be used.

(Other Ultraviolet Absorbers)

In all the aspects, the resin composition of the present disclosure preferably further contains ultraviolet absorbers other than the specific compound (II).

In a case where the resin composition contains other ultraviolet absorbers, the ultraviolet cut properties of the resin composition and the wavelength region of ultraviolet rays that can be cut off can be adjusted.

Examples of those other ultraviolet absorbers that the resin composition can contain are the same as the examples of other ultraviolet absorbers described above regarding the polymerizable composition (I-1) and the polymerizable composition (I-2), and preferable examples thereof are also the same.

It is preferable to select those other ultraviolet absorbers in consideration of the compatibility and affinity with the polymer compound or polymer to be used in combination, and the like.

In a case where the resin composition of the present disclosure contains other ultraviolet absorbers, the content of those other ultraviolet absorbers with respect to the total solid content of the resin composition is preferably 0.01% by mass to 10% by mass, and more preferably 0.01% by mass to 5% by mass.

(Solvent)

The resin composition of the present disclosure may further contain a solvent.

In a case where the resin composition contains a solvent, the viscosity of the resin composition can be appropriately adjusted. For example, in a case where a solvent is added to the resin composition so that the resin composition has fluidity, it is possible to improve workability in forming a resin composition layer by using the resin composition by means of coating or in filling a molding die with the resin composition.

Furthermore, in a case where a solvent is added to the resin composition so that the resin composition has appropriate viscosity, it is possible to perform press molding on the resin composition by disposing the resin composition in a molding die.

As the solvent that the resin composition can contain, it is preferable to select an organic solvent that can dissolve the polymer compound or polymer contained in the resin composition. The usable solvent may be selected from the solvents exemplified above regarding the polymerizable composition (I-1) and the polymerizable composition (I-2), in consideration of the affinity with the polymer compound or polymer, and the like.

As the solvent that the resin composition of the present disclosure can contain, a ketone-based solvent such as methyl ethyl ketone, methyl isobutyl ketone, cyclohexanone, or cyclopentanone, an ester-based solvent such as propyl acetate, butyl acetate, or 1-methoxy-2-propyl acetate, and a halogen-based solvent such as chloroform are preferable.

In a case where the resin composition contains a solvent, the content of the solvent is appropriately selected depending on the physical properties of the target resin composition. Generally, the content of the solvent with respect to the total mass of the resin composition is preferably 20% by mass to 90% by mass, and more preferably 50% by mass to 70% by mass.

[Ultraviolet Cut Film (Second Aspect)]

A second aspect of the ultraviolet cut film of the present disclosure is a cured substance of the polymerizable composition (II) of the present disclosure described above, a cured substance of the resin composition of the present disclosure containing the specific compound (II) and a polymer compound, or a cured substance of a resin composition containing the polymer of the present disclosure.

In the second aspect of the ultraviolet cut film of the present disclosure, the ultraviolet cut film contains the specific compound (II) or a polymer containing a constitutional unit derived from the specific compound (II). In this way, excellent ultraviolet cut properties are obtained, coloration is suppressed, and excellent visible light transmittance is obtained.

There is no particular limitation on the manufacturing method of the cured substance of the resin composition.

In a case where the resin composition contains a solvent, a cured substance can be obtained by removing the solvent.

In a case where the polymer compound is a thermoplastic resin, a cured substance can be obtained by heating and melting the resin composition, molding the resin composition in an appropriate shape, and cooling the resin composition.

In a case where the polymer compound contained in the resin composition is a thermosetting polymer compound, a cured substance can be obtained by filling a molding die with the resin composition and curing the polymer compound by a known method such as heating.

In a case where the polymer compound has a polymerizable group, by additionally incorporating a photopolymerization initiator or a thermal polymerization initiator into the resin composition, it is possible to obtain a cured substance by the application of energy such as light or heat.

In a case where light irradiation is performed as the application of energy, for example, an ultraviolet lamp can be used. The light irradiation dose is preferably in a range of 10 mJ/cm² to 1,000 mJ/cm². In a case where ultraviolet rays are radiated at this irradiation dose, the polymerizable composition layer is suitably cured, which makes it possible to efficiently obtain an ultraviolet cut film as a cured substance.

Furthermore, by a method of cutting the obtained cured substance in addition to the molding method using a molding die, it is possible to process the obtained cured substance into a desired shape.

The second aspect of the ultraviolet cut film is a cured substance of the polymerizable composition (II) of the present disclosure or the resin composition of the present disclosure described above. Therefore, due to the makeup of the polymerizable composition (II), the content of the specific compound (II) in the ultraviolet cut film is preferably within a range of 0.005 mmol (millimoles)/m² to 0.1 mmol/m², and more preferably in a range of 0.01 mmol/m² to 0.05 mmol/m².

The ultraviolet cut film of the second aspect of the present disclosure contains the specific compound (II) or the polymer containing a constitutional unit derived from the specific compound (II). Therefore, the ultraviolet cut film excellently cuts off the wavelength region including at least the long wavelength region of ultraviolet light and the short wavelength region of visible light. As described above, in a case where the polymerizable composition (II) of the present disclosure or the resin composition of the present disclosure further contains other ultraviolet absorbers, the ultraviolet cut film of the second aspect of the present disclosure can be an ultraviolet cut film that can effectively cut off the desired wavelength region of ultraviolet rays.

[Laminate (Second Aspect)]

A second aspect of the laminate of the present disclosure has a support and an ultraviolet cut film which is a cured substance of the polymerizable composition (II) of the present disclosure described above, a cured substance of the resin composition of the present disclosure containing the specific compound (II) and a polymer compound, or a cured substance of a resin composition containing the polymer of the present disclosure.

In the second aspect of the laminate of the present disclosure, the laminate is the same as the laminate described above, except that a polymer containing the specific compound (II) or a constitutional unit derived from the specific compound (II) is used instead of the specific compound (I-1) or the specific compound (I-2). Furthermore, the usable support, the layer constitution that can be adopted for the laminate, and the like are also the same.

[Application of Polymerizable Composition, Resin Composition, Ultraviolet Cut Film, and Laminate]

It is known that a yellow colorant having maximal absorption at a wavelength of 400 nm to 500 nm can cut off blue light in the visible light region. Focusing on the absorption characteristics of compounds, the inventors of the present invention have found that problems can be solved using the aforementioned specific compound (I-1), specific compound (I-2), or specific compound (II), as a compound which has an absorption spectrum where a sharp peak of maximal absorption wavelength appears and has extremely low absorption in a wavelength region longer than the maximal absorption wavelength.

That is, even though a dye has maximal absorption at a wavelength of 400 nm to 500 nm, in a case where the dye has absorption in a longer wavelength region, blue light can be cut off. However, unfortunately, the obtained polymerizable composition and the cured substance thereof have a reddish tone, and the reddish tone such as orange or red is easy to visually recognize.

Therefore, in a case where a compound having absorption in a wavelength region longer than the target ultraviolet rays in the present disclosure is used in an apparatus comprising a display, such as an image display apparatus including a liquid crystal display device and an electroluminescent display or a small terminal including a smartphone and a tablet terminal, the color reproducibility of a display image seen on the display deteriorates. Furthermore, in a case where a compound having absorption in a wavelength region longer than ultraviolet rays in the present disclosure is used for a spectacle lens, a contact lens, and the like, unfortunately, the lens is colored yellow or red and looks unattractive. These problems are solved by the polymerizable composition (I-1), the polymerizable composition (I-2), the polymerizable composition (II), and the resin composition containing the specific compound (I-1), the specific compound (I-2), or the specific compound (II).

Accordingly, the polymerizable composition (I-1), polymerizable composition (I-2), polymerizable composition (II), and resin composition of the present disclosure and the ultraviolet cut film and laminate as an example of the usage aspects thereof are suitably used for various uses that require ultraviolet cut properties.

Specific examples of the uses include an ultraviolet cut material for apparatuses comprising a display, such as an image display apparatus including a liquid crystal display device and an electroluminescent display and a small terminal including a smartphone and a tablet terminal, an ultraviolet cut material for a spectacle lens, an ultraviolet cut material for a contact lens, and the like.

In addition, examples of other uses include an intraocular lens, window glass, plastics, fiber, paper, paint, ink, cosmetics, and the like. The polymerizable composition, resin composition, ultraviolet cut film, and laminate of the present disclosure can be used in various fields in need of cutting off ultraviolet rays.

The polymerizable composition (I-1), polymerizable composition (I-2), and polymerizable composition (II) of the present disclosure are suitably used for manufacturing contact lenses, intraocular lenses, and the like. By thoroughly stirring and mixing the polymerizable composition of the present disclosure, preferably the polymerizable composition containing a polymerization initiator and other components, injecting the polymerizable composition into a molding die, and performing at least either photocuring or thermal curing, it is possible to manufacture a contact lens, an intraocular lens, and the like.

The contact lens, intraocular lens, or the like obtained from at least one of the polymerizable composition (I-1), polymerizable composition (I-2), or polymerizable composition (II) of the present disclosure preferably has maximal absorption in a wavelength range of 380 nm to 430 nm and is inhibited from experiencing elution, bleed out, and the like of the specific compound (I-1), the specific compound (I-2), or the specific compound (II). Therefore, the contact lens, the intraocular lens, or the like has excellent ultraviolet cut properties and is excellently inhibited from experiencing transparency reduction with the passage of time.

### EXAMPLES

Hereinafter, the present disclosure will be more specifically described with reference to examples, but the present disclosure is not limited to the following examples as long as the gist of the present disclosure is maintained.

Unless otherwise specified, “%” and “part” in the following examples are based on mass.

Hereinafter, in some cases, a methyl group will be abbreviated to “Me”, an ethyl group will be abbreviated to “Et”, an acetyl group will be abbreviated to “Ac, and a phenyl group will be abbreviated to “Ph”.

### Example 1

### Manufacturing of Exemplary Compound 1

An exemplary compound 1 was manufactured according to the following scheme.

At room temperature, 0.7 g of triethylamine was added to a mixture of 2.5 g of 3-methyl-2-(2-N-acetylanilinovinyl)benzoxazolium iodide, 1.4 g of 2-methacryloyloxyethyl cyanoacetate, and 6 mL (milliliters) of acetonitrile. The reaction mixture was stirred at room temperature for 3 hours, and 6 mL of deionized water was added thereto. After stirring for 1 hour, the precipitated crystals were collected by filtration and washed with a mixed solution of acetonitrile/deionized water=1/1 (vol). Isopropyl alcohol (18 mL) was added to the obtained crystals, and the mixture was heated under reflux. The mixture was cooled to room temperature, collected by filtration, and dried, thereby obtaining 1.7 g of the exemplary compound 1.

“Room temperature” in synthesizing the exemplary compound means “room temperature not being particularly controlled” that depends on the season, environment, and the like. In Examples of the present disclosure, the room temperature is a temperature range of 18° C. to 27° C.

In a case where the room temperature is in the temperature range of 18° C. to 27° C., the manufacturing of an exemplary compound and the like can be performed without any problem.

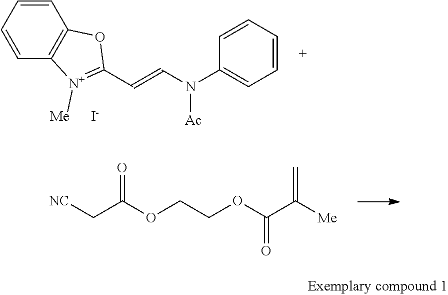 [SMILES: CC(=O)N(/C=C/c1oc2ccccc2[n+]1C)c1ccccc1 ; [I-] ; C=C(C)C(=O)OCCOC(=O)CC#N]

Regarding the exemplary compound 1, the results of NMR spectroscopy, the maximal absorption wavelength (described as λmax) measured by the method described above, and the ratio ([ε(440)/ε(405)]) of the absorption at a wavelength of 440 nm to absorption at a wavelength of 405 nm are shown below.

¹H-NMR (CDCl₃) δ: 8.47 (d, 1H), 7.37 (d, 1H), 7.31 (dd, 1H), 7.23 (dd, 1H), 7.10 (d, 1H), 6.17 (br, 1H), 5.60 (br, 1H), 5.40 (d, 1H), 4.50-4.39 (m, 4H), 3.53 (s, 3H), 1.97 (s, 3H)

λmax 412 nm, ε73,200 (ethyl acetate)

[ε(440)/ε(405)]=0.0144

As described above, it has been confirmed that the exemplary compound 1 has maximal absorption in a wavelength range of 390 nm to 430 nm and has small absorption at a longer wavelength of 440 nm.

### Example 2

### Manufacturing of Exemplary Compound 9

An exemplary compound 9 was manufactured according to the following scheme.

At room temperature, 0.8 g of triethylamine was added to a mixture of 2.9 g of 3-methyl-2-(2-N-acetylanilinovinyl)benzoxazolium iodide, 1.6 g of 2-methacryloyloxyethyl acetoacetate, and 7 mL of acetonitrile. The reaction mixture was stirred at room temperature for 3 hours, and 7 mL of deionized water was added thereto. After stirring for 1 hour, the precipitated crystals were collected by filtration and washed with a mixed solution of acetonitrile/deionized water=1/1 (vol). The obtained crystals were recrystallized from 14 mL of isopropyl alcohol, thereby obtaining 1.4 g of the exemplary compound 9 as a geometric isomer mixture.

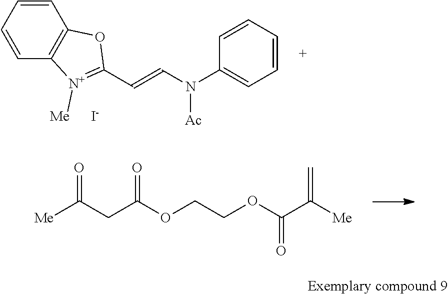 [SMILES: CC(=O)N(/C=C/c1oc2ccccc2[n+]1C)c1ccccc1 ; [I-] ; C=C(C)C(=O)OCCOC(=O)CC(C)=O]

Regarding the exemplary compound 9, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 8.58 and 8.41 (d, 1H), 7.35 (d, 1H), 7.28 (dd, 1H), 7.21 (dd, 1H), 7.07 (d, 1H), 6.93 and 6.23 (d, 1H), 6.17 (br., 1H), 5.60 (br, 1H), 4.48 (m, 4H), 3.52 (s, 3H), 2.48 (s, 3H), 1.97 (s, 3H)

λmax 423 nm, ε72,900 (ethyl acetate)

[ε(440)/ε(405)]=0.296

As described above, it has been confirmed that the exemplary compound 9 has maximal absorption in a range of 390 nm to 430 nm and has small absorption at a longer wavelength of 440 nm.

### Example 3

### Manufacturing of Exemplary Compound 31

An exemplary compound 31 was manufactured according to the following scheme.

At room temperature, 0.8 g of triethylamine was added to a mixture of 3.0 g of 3-ethyl-2-(2-N-acetylanilinovinyl)benzoxazolium iodide, 2.4 g of 2-[2-(phenylsulfonyl)acetoxy]ethyl methacrylate, and 7 mL of acetonitrile. The reaction mixture was stirred at room temperature for 12 hours, and 10.5 mL of deionized water was added thereto. The precipitated crystals were collected by filtration and washed with deionized water. The obtained crystals were recrystallized from 7 mL of acetonitrile, thereby obtaining 1.8 g of the exemplary compound 31.

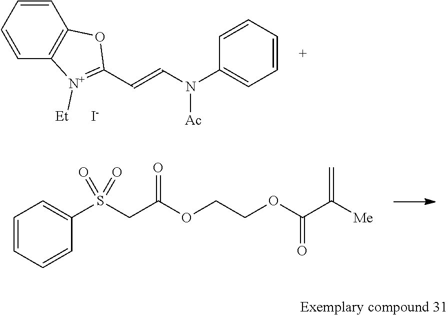 [SMILES: CC[n+]1c(/C=C/N(C(C)=O)c2ccccc2)oc2ccccc21 ; [I-] ; C=C(C)C(=O)OCCOC(=O)CS(=O)(=O)c1ccccc1]

Regarding the exemplary compound 31, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 8.77 (d, 1H), 7.93 (d, 2H), 7.50-7.37 (m, 4H), 7.35-7.23 (m, 2H), 7.12 (dd, 1H), 6.45 (d, 1H), 6.14 (br., 1H), 5.60 (br, 1H), 4.32 (m, 2H), 4.18 (m, 2H), 4.00 (q, 2H), 1.94 (s, 3H), 1.43 (t, 3H)

λmax 405 nm, ε88,600 (ethyl acetate)

[ε(440)/ε(405)]=0.0044

As described above, it has been confirmed that the exemplary compound 31 has maximal absorption in a wavelength range of 390 nm to 430 nm and has small absorption at a longer wavelength of 440 nm.

### Example 4

### Manufacturing of Mixture of Exemplary Compound 33 and Exemplary Compound 34

Exemplary compounds 33 and 34 were manufactured according to the following scheme.

At room temperature, 0.8 g of triethylamine was added to a mixture of 3.0 g of 3-ethyl-2-(2-N-acetylanilinovinyl)benzoxazolium iodide, 2.5 g of an isomer mixture of 1-[2-(phenylsulfonyl)acetoxy]propan-2-yl methacrylate and 2-[2-(phenylsulfonyl)acetoxy]propan-1-yl methacrylate, and 7 mL of acetonitrile. The reaction mixture was stirred at room temperature for 12 hours, and 50 mL of deionized water was added thereto. The mixture was extracted with 50 mL of ethyl acetate, washed with a saline solution, then dried over magnesium sulfate, and filtered. BHT was added to the filtrate, and the mixture was concentrated under reduced pressure. The residue was purified by silica gel chromatograph, thereby obtaining 2.4 g of an isomer mixture of the exemplary compound 33 and the exemplary compound 34.

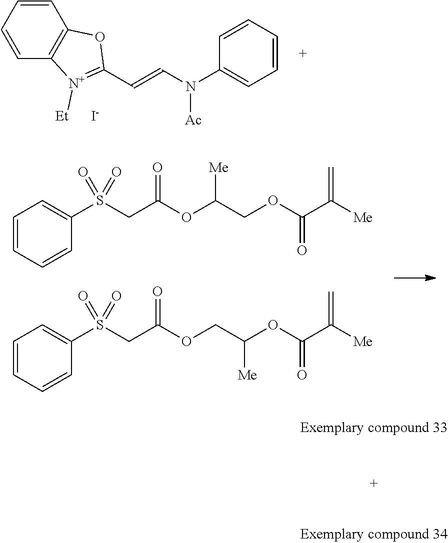 [SMILES: CC[n+]1c(/C=C/N(C(C)=O)c2ccccc2)oc2ccccc21 ; [I-] ; C=C(C)C(=O)OCC(C)OC(=O)CS(=O)(=O)c1ccccc1 ; C=C(C)C(=O)OC(C)COC(=O)CS(=O)(=O)c1ccccc1]

Regarding the mixture of the exemplary compound 33 and the exemplary compound 34, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 8.76 and 8.54 (d, 1H), 7.92 (d, 2H), 7.54-7.37 (m, 4H), 7.35-7.22 (m, 2H), 7.12 (d, 1H), 6.70 and 6.47 (d, 1H), 6.08 (br., 1H), 5.54 (br, br, 1H), 5.25-4.98 (m, 1H), 4.28-3.91 (m, 4H), 1.97 and 1.89 (s, 3H), 1.47-1.40 (m, 3H), 1.20-1.13 (m, 3H)

λmax 405 nm, ε87,200 (ethyl acetate)

[ε(440)/ε(405)]=0.0043

As described above, it has been confirmed that the mixture of the exemplary compound 33 and the exemplary compound 34 as an isomer thereof has maximal absorption in a wavelength range of 390 nm to 430 nm and has small absorption at a longer wavelength of 440 nm.

### Example 5

### Manufacturing of Mixture of Exemplary Compound 49 and Exemplary Compound 50

Exemplary compounds 49 and 50 were manufactured according to the following scheme.

At room temperature, 0.6 g of triethylamine was added to a mixture of 2.1 g of 3-methyl-2-(2-N-acetylanilinovinyl)benzoxazolium iodide, 1.5 g of an isomer mixture of 4-vinylbenzyl cyanoacetate and 3-vinylbenzyl cyanoacetate, and 5 mL of acetonitrile. The reaction mixture was stirred at room temperature for 12 hours, and 5 mL of deionized water was added thereto. The precipitated product was collected by filtration and washed with a mixed solution of acetonitrile/distilled water=1/1. The product was recrystallized using 5 mL of acetonitrile, thereby obtaining 0.3 g the exemplary compound 49 and the exemplary compound 50 as an isomer mixture.

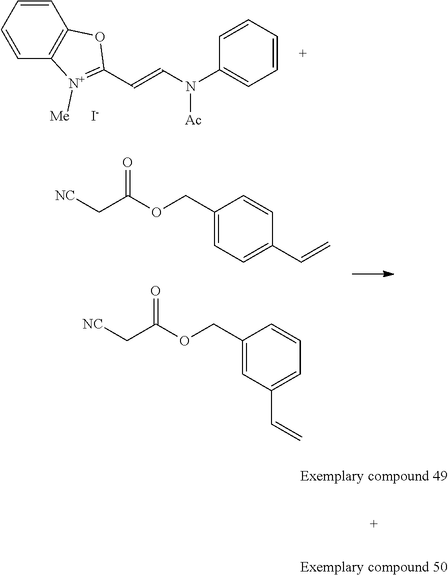 [SMILES: CC(=O)N(/C=C/c1oc2ccccc2[n+]1C)c1ccccc1 ; [I-] ; C=Cc1ccc(COC(=O)CC#N)cc1 ; C=Cc1cccc(COC(=O)CC#N)c1]

Regarding the mixture of the exemplary compound 49 and the exemplary compound 50, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 8.48 (d, 1H), 7.49-7.38 (m, 3H), 7.37-7.19 (m, 4H), 7.09 (d, 1H), 6.78-6.67 (m, 1H), 5.82-5.72 (m, 1H), 5.40 (d, 1H), 5.29-5.22 (m, 1H), 5.26 (s, 2H), 3.52 (s, 3H)

λmax 413 nm, ε68,500 (ethyl acetate)

[ε(440)/ε(405)]=0.0167

As described above, it has been confirmed that the mixture of the exemplary compound 49 and the exemplary compound 50 as an isomer thereof has maximal absorption in a wavelength range of 390 nm to 430 nm and has small absorption at a longer wavelength of 440 nm.

### Example 6

### Manufacturing of Mixture of Exemplary Compound 59 and Exemplary Compound 60

Exemplary compounds 59 and 60 were manufactured according to the following scheme.

At room temperature, 0.5 g of triethylamine was added to a mixture of 1.7 g of 3-methyl-2-(2-N-acetylanilinovinyl)benzoxazolium iodide, 2.0 g of an isomer mixture of 4-vinylbenzyl phenylsulfonyl acetate and 3-vinylbenzyl phenylsulfonyl acetate, and 4 mL of acetonitrile. The reaction mixture was stirred at room temperature for 12 hours, and 4 mL of deionized water was added thereto. The precipitated product was collected by filtration and washed with a mixed solution of acetonitrile/distilled water=1/1. The product was recrystallized using 4 mL of acetonitrile, thereby obtaining 1.1 g the exemplary compounds 59 and 60 as an isomer mixture.

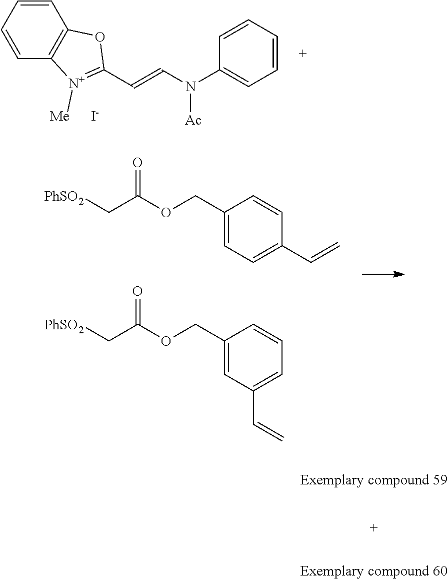

Regarding the mixture of the exemplary compound 59 and the exemplary compound 60, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 8.75 (d, 1H), 7.84 (m, 2H) 7.49-7.39 (m, 2H), 7.36-7.21 (m, 6H), 7.16-7.05 (m, 3H), 6.69 (m, 1H), 6.39 (d, 1H), 5.76 (d, 1H), 5.27 (d, 1H), 5.10 and 5.08 (s, 2H), 3.43 (s, 3H)

λmax 405 nm, ε90,000 (ethyl acetate)

[ε(440)/ε(405)]=0.0034

As described above, it has been confirmed that the mixture of the exemplary compound 59 and the exemplary 60 as an isomer thereof has maximal absorption in a wavelength range of 390 nm to 430 nm and has small absorption at a longer wavelength of 440 nm.

### Comparative Example 1

The following comparative compound C-1 was synthesized according to Example 1 described in WO2019/073869A.

Under cooling with ice water, 1.7 g of diisopropylcarbodiimide was added to a mixture of 1.6 g of 2-hydroxyethyl methacrylate, 1.1 g of cyanoacetic acid, 0.1 g of 4-dimethylaminopyridine, and 12 mL of toluene. The reaction mixture was stirred at room temperature for 3 hours, then filtered, and added to a mixture of 2.6 g of 1-methyl-2-anilinovinylpyrrolinium iodide, 1.0 g of acetic anhydride, and 4 mL of acetonitrile. Diisopropylethylamine (1.3 g) was added to the mixture being stirred at room temperature, and the mixture was stirred at room temperature overnight. Distilled water (20 mL) and 20 mL of hexane were added to the reaction mixture, and the precipitated powder was filtered and sequentially washed with distilled water and hexane. The power was recrystallized using 16 mL of isopropyl alcohol, thereby obtaining 0.9 g of the following comparative compound C-1.

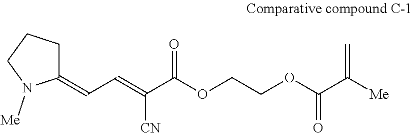 [SMILES: C=C(C)C(=O)OCCOC(=O)/C(C#N)=C/C=C1\CCCN1C]

Regarding the comparative compound C-1, the results of NMR spectroscopy, the maximal absorption wavelength (described as λmax) measured by the method described above, and the ratio of the absorption at a wavelength of 440 nm to absorption at a wavelength of 405 nm are shown below.

¹H-NMR (CDCl₃) δ: 7.92 (d, 1H), 6.16 (br, 1H), 5.58 (br, 1H), 5.55 (d, 1H), 4.47-4.37 (m, 4H), 3.66 (m, 2H), 3.06 (s, 3H), 3.03 (m, 2H), 2.10 (m, 2H), 1.95 (s, 3H)

λmax 388 nm, ε53,700 (ethyl acetate)

[ε(440)/ε(405)]=0.0042

As described above, the comparative compound C-1 has a maximal absorption wavelength less than 390 nm. Presumably, this compound may have insufficient absorption in a long wavelength region of ultraviolet rays and a short wavelength region of visible light.

### Example 7 and Example 8

(1. Preparation of Polymerizable Composition)

By using the exemplary compound 31 as the specific compound (II), 2-hydroxyethyl methacrylate as a polymerizable compound having the following structure, Omnirad 819 (former name: IRGACURE819, manufactured by BASF SE) as a polymerization initiator, and RUVA-93 (trade name, 2-[2′-hydroxy-5′-(methacryloyloxyethyl)phenyl-2H-benzotriazole, manufactured by Otsuka Chemical Co., Ltd.) as other ultraviolet absorbers at the content shown in the following Table 1, a polymerizable composition of Example 7 and a polymerizable composition of Example 8 were prepared. In Table 1, the polymerization initiator and other ultraviolet absorbers are described as the names of commercially available products. “-” in Table 1 means that the example does not contain the corresponding component.

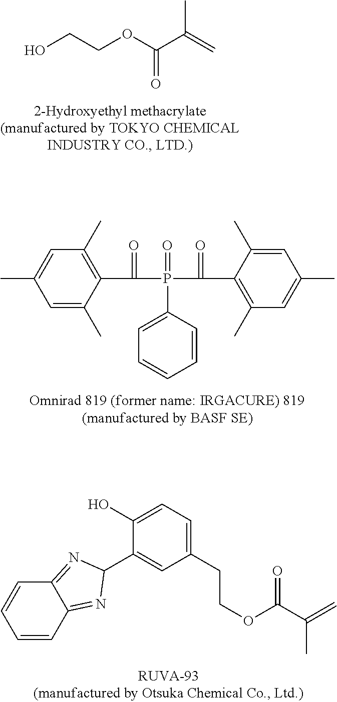

TABLE 1
[% by mass]

| Makeup of polymerizable composition | | Example 7 | Example 8 |
|---|---|---|---|
| Polymerizable | 2-Hydroxyethyl methacrylate | 99.1 | 97.1 |
| compound | | | |
| Specific | Exemplary compound 31 | 0.5 | 0.5 |
| compound (II) | | | |
| Other ultraviolet | RUVA-93 | — | 2 |
| absorbers | | | |
| Polymerization | Omnirad 819 | 0.4 | 0.4 |
| initiator | (IRGACURE 819) | | |
| | Total | 100 | 100 |

(2. Manufacturing Ultraviolet Cut Film as Cured Substance of Polymerizable Composition)

Each polymerizable composition is sandwiched between crown glass plates having a thickness of 1 mm, and irradiated with light at 1.0 J/cm² (2.5 mW/cm²) by using a light irradiation device (EXECURE 3000, HOYA CANDEO OPTRONICS CORPORATION), thereby preparing an ultraviolet cut film 1 and an ultraviolet cut film 2 in which each polymerizable composition was sandwiched between glass plates. The distance between the glass plates was adjusted so that the ultraviolet cut film had a thickness of 50 μm.

(3. Evaluation of Ultraviolet Cut Film)

(3-1. Evaluation of Light Transmittance)

By using the ultraviolet cut film 1 and the ultraviolet cut film 2 sandwiched between glass plates prepared under the above conditions, the transmittance at a wavelength of 300 nm to 800 nm was measured. The results are shown in the following table 2.

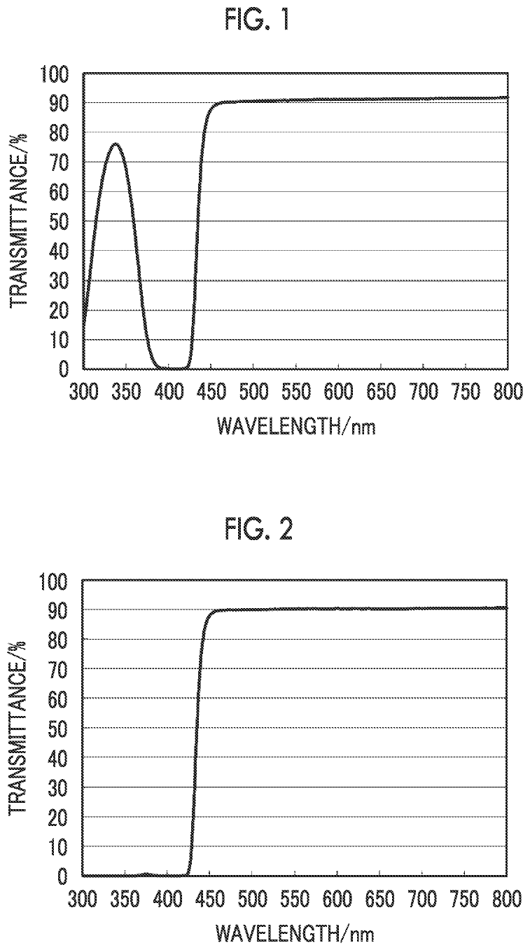

FIG. 1 shows the transmittance spectrum of the ultraviolet cut film 1 at 300 nm to 800 nm. FIG. 2 shows the transmittance spectrum of the ultraviolet cut film 2 at 300 nm to 800 nm.

TABLE 2

| | Ultraviolet | Ultraviolet |
|---|---|---|
| | cut film 1 | cut film 2 |
| Transmittance (wavelength) | (Example 7) | (Example 8) |
| Transmittance (340 nm) | 76% | 0% |
| Transmittance (370 nm) | 21% | 0% |
| Transmittance (400 nm) | 0% | 0% |
| Transmittance (430 nm) | 19% | 15% |
| Transmittance (450 nm) | 88% | 88% |

As is evident from Table 2 and FIGS. 1 and 2, both the ultraviolet cut film 1 and ultraviolet cut film 2 excellently cut off ultraviolet rays in a wavelength range of 390 nm to 430 nm and has excellent light transmittance in a wavelength range of 440 nm or more. In addition, it has been confirmed that the ultraviolet cut film 2 additionally containing other ultraviolet absorbers excellently cuts off the shorter wavelength region and the long wavelength region of ultraviolet rays, specifically, the ultraviolet rays in a broad wavelength range of around 300 nm to 430 nm.

(3-2. Evaluation of Storage Stability)

The ultraviolet cut film 1 and the ultraviolet cut film 2 were stored under the conditions of 40° C. and a humidity of 50% RH for 1 week, and then left at room temperature for 1 day.

Then, the ultraviolet cut film 1 and the ultraviolet cut film 2 were visually observed. As a result, none of the specific compound (II) and other ultraviolet absorbers were found to cause bleed out and the like. Therefore, it has been confirmed that the ultraviolet cut films as cured substances of Example 7 and Example 8 are inhibited from experiencing bleed out and the like of the specific compound (II) and other ultraviolet absorbers even under severe conditions.

### Example 9

### Manufacturing of Copolymer of Exemplary Compound 31 and Methyl Methacrylate

A mixture of 100 mg of the exemplary compound 31 obtained in Example 3, 9.9 g of methyl methacrylate, and 40.0 g of propylene glycol monomethyl ether acetate was stirred at 80° C. for 30 minutes under a nitrogen stream.

V-601 (136 mg, manufactured by FUJIFILM Wako Pure Chemical Corporation) was added to this solution, and the mixture was stirred at 80° C. for 4 hours under a nitrogen stream. Furthermore, V-601 was added to 23 mg of the reaction mixture, and the mixture was further stirred at 90° C. for 2 hours under a nitrogen stream. The reaction mixture was cooled to room temperature and added to 200 mL of hexane with stirring. After 2 hours of stirring at room temperature, the precipitated solids were collected by filtration and washed with hexane.

Hexane (100 mL) was added to the obtained solids, and the mixture was heated under reflux. The mixture was cooled to room temperature, then collected by filtration, and dried, thereby obtaining 8.3 g of an exemplary polymer A which is the polymer of the present disclosure.

The number average molecular weight of the obtained copolymer was 15,600 (polystyrene-equivalent molecular weight).

The obtained exemplary polymer A (40 mg) was dissolved in 100 mL of chloroform, and the absorption spectrum was measured (optical path length: 1 cm). λmax was 409 nm, and the absorbance was 0.795.

### Example 10

### Manufacturing of Copolymer of Exemplary Compound 31 and 2-[5-(2-methacryloyloxyethyl)-2-hydroxy]phenyl-2h-benzo[d][1,2,3]triazole, and methyl methacrylate

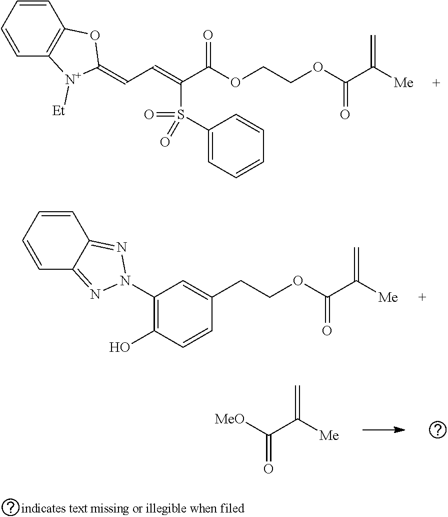

A mixture of 100 mg of the exemplary compound 31 obtained in Example 3, 394 mg of 2-[5-(2-methacryloyloxyethyl)-2-hydroxy]phenyl-2H-benzo[d][1,2,3]triazole, 9.5 g of methyl methacrylate, and 40.0 g of propylene glycol monomethyl ether acetate was stirred at 80° C. for 30 minutes under a nitrogen stream.

V-601 (169 mg, manufactured by FUJIFILM Wako Pure Chemical Corporation) was added to this solution, and the mixture was stirred at 80° C. for 2 hours under a nitrogen stream. V-601 (81 mg) was further added thereto, and the obtained solution was stirred at 80° C. under a nitrogen stream for 2 hours, and then stirred at 90° C. for 2 hours under a nitrogen stream. The reaction mixture was cooled to room temperature and added to 200 mL of hexane with stirring. After 2 hours of stirring at room temperature, the precipitated solids were collected by filtration and washed with hexane.

Isopropyl alcohol (30 mL) and 70 mL of hexane were added to the obtained solids, and the mixture was stirred at room temperature for 4 hours. Then, solids were collected by filtration and dried, thereby obtaining 8.6 g of an exemplary polymer B which is the polymer of the present disclosure. The exemplary polymer B is a copolymer of the exemplary compound 31 which is the specific compound (II), a benzotriazole-based ultraviolet absorber which is another ultraviolet absorber, and a polymerizable compound.

The number average molecular weight of the obtained exemplary polymer B was 12,800 (polystyrene-equivalent molecular weight).

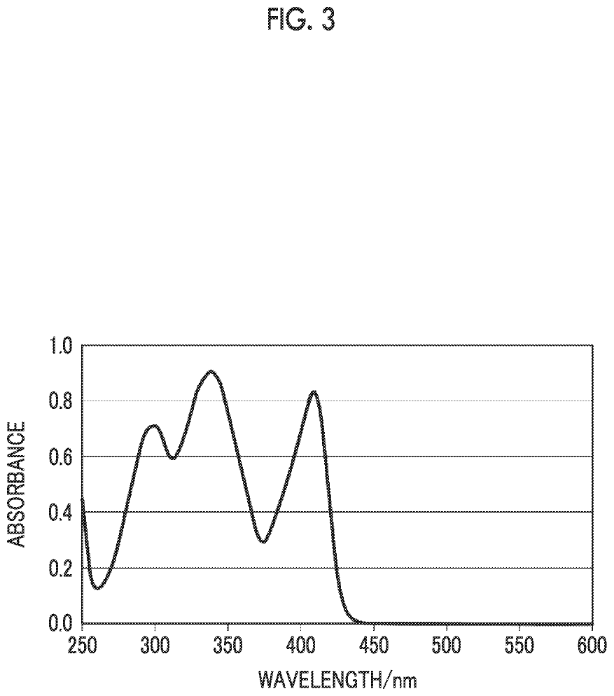

The obtained exemplary polymer B (40 mg) was dissolved in 100 mL of chloroform, and the absorption spectrum was measured. The results are shown in FIG. 3.

As is evident from the absorption spectrum of FIG. 3, the exemplary polymer B has maximal absorption at a wavelength of 410 nm due to the exemplary compound 31 and at a wavelength of around 300 nm to 350 nm due to the benzotriazole-based ultraviolet absorber, cuts off a broad ultraviolet region that spans a shorter wavelength region and a long wavelength region of ultraviolet rays, and has excellent transmittance in a wavelength range of 440 nm or more.

### Example 11

### Preparation of Resin Composition Containing Exemplary Compound 31, Polymer Compound, and Solvent

The exemplary compound 31 (14.2 mg) obtained in Example 3, 1.1 g of a polymer compound (DIANAL BR-80 (PMMA-based polymer containing 60% by mass or more of methyl methacrylate as a monomer unit, Mw: 95,000, acid value: 0 mg KOH/g, manufactured by Mitsubishi Chemical Corporation.), and 7.6 g of a solvent (chloroform) were stirred and mixed together at room temperature for 30 minutes, thereby obtaining a resin composition of Example 11.

(Manufacturing of Laminate)

A transparent support (glass plate) was spin-coated with the obtained resin composition, and dried at 100° C. for 2 minutes, thereby obtaining a laminate having an ultraviolet cut film as a cured substance of the resin composition on the transparent support. The film thickness of the ultraviolet cut film after drying was 10 μm.

(Evaluation of Laminate)

The laminate measured by the same method as described above had a transmittance of 1% at a wavelength of 400 nm and a transmittance of 88% at a wavelength of 440 nm.

This result tells that the laminate of Example 11 has ultraviolet cut properties and excellent transparency.

### Example 12

### Preparation of Resin Composition Containing Exemplary Polymer B Containing Constitutional Unit Derived from Exemplary Compound 31 and Solvent

The exemplary polymer B (1.1 g) obtained in Example 10 and 7.6 g of a solvent (chloroform) were stirred and mixed together at room temperature for 30 minutes, thereby obtaining a resin composition of Example 12.

(Manufacturing of Laminate)

The same transparent support as that used in Example 11 was spin-coated with the obtained resin composition, and dried at 100° C. for 2 minutes, thereby obtaining a laminate having an ultraviolet cut film as a cured substance of the resin composition on the transparent support.

(Evaluation of Laminate)

The laminate measured by the same method as described above had a transmittance of 2% or less in a wavelength range of 300 nm to 400 nm and a transmittance of 88% at a wavelength of 440 nm.

This result tells that the laminate of Example 12 has ultraviolet cut properties and excellent transparency.

### Example 13

### Preparation and Evaluation of Polymerizable Composition Containing Exemplary Compound I-1-5

The exemplary compound I-1-5 (0.48 g), 100 mL of methyl methacrylate as a polymerizable compound, and 0.67 mg of a polymerization initiator Omnirad (former name: IRGACURE) 819 were mixed together, thereby preparing a polymerizable composition.

The maximal absorption wavelength of the polymerizable composition measured by the method described above was 440 nm.

### Example 14

### Preparation and Evaluation of Polymerizable Composition Containing Exemplary Compound I-1-13

The exemplary compound I-1-13 (0.67 g), 100 mL of methyl methacrylate as a polymerizable compound, and 0.67 mg of a polymerization initiator Omnirad (former name: IRGACURE) 819 were mixed together, thereby preparing a polymerizable composition.

The maximal absorption wavelength of the polymerizable composition measured by the method described above was 436 nm.

The disclosure of Japanese Patent Application No. 2019-149171 filed Aug. 15, 2019 is incorporated into the present disclosure by reference.

All of documents, patent applications, and technical standards described in the present disclosure are incorporated into the present disclosure by reference to approximately the same extent as a case where it is specifically and respectively described that the respective documents, patent applications, and technical standards are incorporated by reference.

What is claimed is:

1. A polymerizable composition comprising:
a compound represented by General Formula (I-1); and
a polymerizable compound, 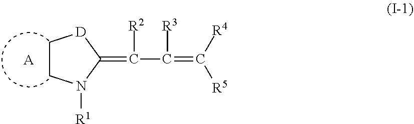
in General Formula (I-1), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group,
D represents an oxygen atom, a sulfur atom, or N-E, and E represents an alkyl group, A represents a 5- or 6-membered saturated or unsaturated ring, and the 5- or 6-membered ring may be further condensed.

2. A polymerizable composition comprising:
a compound represented by General Formula (I-2); and
a polymerizable compound, 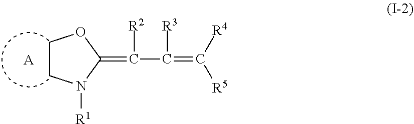
in General Formula (I-2), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, and the 5- or 6-membered ring may be further condensed.

3. The polymerizable composition according to claim 2,
wherein R⁴ and R⁵ in General Formula (I-2) each independently represent a cyano group, an alkylsulfonyl group, an arylsulfonyl group, an alkoxycarbonyl group, a carbamoyl group, an alkylcarbonyl group, or an arylcarbonyl group.

4. The polymerizable composition according to claim 2,
wherein A in General Formula (I-2) represents a benzene ring or a naphthalene ring.

5. The polymerizable composition according to claim 2,
wherein the compound represented by General Formula (I-2) has absorption maximum in a wavelength range of 390 nm to 430 nm in ethyl acetate.

6. The polymerizable composition according to claim 2, further comprising:
an ultraviolet absorber other than the compound represented by General Formula (I-2).

7. An ultraviolet cut film which is a cured substance of the polymerizable composition according to claim 1.

8. A laminate comprising:
a support; and
the ultraviolet cut film according to claim 7.

9. A compound represented by General Formula (II), 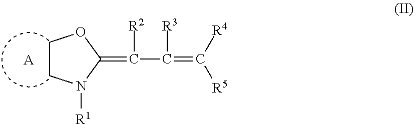
in General Formula (II), R¹ represents a hydrogen atom, an alkyl group, or an aryl group, R² and R³ each independently represent a hydrogen atom, an alkyl group, an aryl group, or a cyano group, R⁴ and R⁵ each independently represent an electron-withdrawing group, A represents a 5- or 6-membered saturated or unsaturated ring, the 5- or 6-membered ring may be further condensed,
at least one of R¹, R², R³, R⁴, R⁵, or A contains a substituent selected from the group consisting of General Formula (III) and General Formula (IV), and in a case where at least one of R¹, R², R³, R⁴, or R⁵ contains a substituent selected from the group consisting of General Formula (III) and General Formula (IV), and at least one of R¹, R², R³, R⁴, or R⁵ may be a substituent selected from the group consisting of General Formula (III) and General Formula (IV) 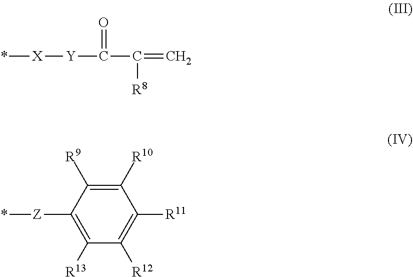
in General Formula (III), X represents a single bond or an alkylene group, Y represents a single bond, —O—, or —NR¹⁴—, R¹⁴ represents a hydrogen atom or an alkyl group, R⁸ represents a hydrogen atom or an alkyl group, and * represents a binding position,
in General Formula (IV), R⁹, R¹⁰, R¹¹, R¹², and R¹³ each independently represent a hydrogen atom, a halogen atom, an alkyl group, or an alkoxy group, Z represents a single bond or an alkylene group, * represents a binding position, and at least one of R⁹, R¹⁰, R¹¹, R¹², or R¹³ represents a vinyl group.

10. The compound according to claim 9,
wherein R⁴ and R⁵ in General Formula (II) each independently represent a cyano group, an alkylsulfonyl group, an arylsulfonyl group, an alkoxycarbonyl group, a carbamoyl group, an alkylcarbonyl group, or an arylcarbonyl group.

11. The compound according to claim 9,
wherein A in General Formula (II) represents a benzene ring or a naphthalene ring.

12. The compound according to claim 9 that has absorption maximum in a wavelength range of 390 nm to 430 nm in ethyl acetate.

13. A polymer comprising:
a constitutional unit derived from the compound according to claim 9.

14. A polymerizable composition comprising:
the compound according to claim 9.

15. The polymerizable composition according to claim 14, further comprising:
an ultraviolet absorber other than the compound represented by General Formula (II).

16. A resin composition comprising:
the compound according to claim 9; and
a polymer compound.

17. A resin composition comprising:
the polymer according to claim 13.

18. The resin composition according to claim 16, further comprising:
an ultraviolet absorber other than the compound represented by General Formula (II).

19. An ultraviolet cut film which is a cured substance of the polymerizable composition according to claim 14.

20. A laminate comprising:
a support; and
the ultraviolet cut film according to claim 19.
